(19) United States
(12) Patent Application Publication
(10) Pub. No.: US 2023/0314450 A1
(43) Pub. Date: Oct. 5, 2023

(54) METHOD FOR DETECTING A ß-SHEET AGGREGATE FORM OF A PROTEIN FORMING ß-SHEET AGGREGATES

(71) Applicant: CISBIO BIOASSAYA, Codolet (FR)

(72) Inventors: Eric J. TRINQUET, Pont-Saint-Esprit (FR); Alexis G. AQUILINA, Bouillargues (FR); Maud E. PRATLONG, Montpezat (FR)

(73) Assignee: CISBIO BIOASSAYA, Codolet (FR)

(21) Appl. No.: 17/925,981

(22) PCT Filed: May 17, 2021
(86) PCT No.: PCT/FR2021/050854

§ 371 Date: Nov. 17, 2022

(30) Foreign Application Priority Data
May 18, 2020 (FR) FR2004948

(51) Int. Cl. G01N 33/68; G01N 33/543

(52) U.S. Cl. CPC G01N 33/6896; G01N 33/543

(57) ABSTRACT

The invention relates to an in-vitro method for detecting in a sample a β-sheet aggregate form of a protein forming β-sheet aggregates (PAPβ), comprising a step of adjusting the pH of a sample likely to contain PAPβ at a pH ranging from 9.7 to 13.2 in order to separate out all or one portion of the PAPβ in order to obtain a β-sheet non-aggregate form of the protein forming β-sheet aggregates (PNAPβ) and measuring the PNAPβ content with an appropriate immunological method at a pH ranging from 6 to 9.

### TECHNICAL FIELD

The invention relates to an in vitro method for detecting in a sample a β-sheet aggregate form of a protein forming β-sheet aggregates (PAU) comprising a step of disaggregating a PAFβ in order to obtain a β-sheet non-aggregate form of the protein forming β-sheet aggregates (PNAFβ) and a step of measuring the PNAFβ content.

### PRIOR ART

The accumulation of often misfolded proteins leads to their aggregation in the cell, causing cellular dysregulations, characteristic of certain neurodegenerative pathologies in particular. β amyloid peptides and more particularly β 1-42 amyloid peptide have been widely studied for the formation of fibrils containing β sheets then of plates in Alzheimer's disease. The aggregation process of β amyloid peptides is well characterized and this process depends on a number of factors in particular their concentration, pH and temperature. Many other proteins, such as RNA and DNA binding proteins, that contain a prion-like domain can also form prion-like aggregates [1]. Proteins containing a prion-like domain are proteins forming β-sheet aggregates (PFβ).

The prion-like domains contain hydrophobic amino acids which promote the formation of fibrils rich in β sheets. The β-sheet aggregate forms of a protein forming β-sheet aggregate (PAFβ) are not soluble in water at neutral pH.

The current diagnosis of these neurodegenerative diseases induced by PAFβ is most often based on imaging, which completes the clinical and neuropsychological examination. It is a cumbersome and expensive procedure that requires several diagnosis steps.

Various diagnosis techniques allowing to demonstrate or quantify the aggregation or oligomerization of PFβ in a biological sample have also been described in the literature.

Structural analysis techniques have allowed to highlight the different levels of aggregation of certain PFβ, in particular β amyloid peptides and TAU or TDP-43 proteins: electron microscopy, atomic force microscopy, Cryo EM, Circular Dichroism, Nuclear Magnetic Resonance, X-ray diffraction. However, these techniques make it difficult to measure the content, even relatively, of aggregates present in a sample. The possible effect of compounds on the level of aggregation (in particular the inhibition of aggregation) of a PFβ cannot therefore really be studied by these techniques.

Techniques capable of separating proteins according to their molecular weight allow to discriminate between high molecular weight PAFβ and low molecular weight PNAFβ. Some of them also allow to discriminate between aggregates of different sizes. Mention may be made, for example, of polyacrylamide gel electrophoresis (PAGE and SDS-PAGE) associated or not with detection by Western Blot. The experimental conditions currently described in these techniques can, however, distort the measurement of the level of aggregation. This is particularly the case for SDS-PAGE in which the combined use of a detergent and a reducing agent (DTT, beta-mercaptoethanol or TCEP) can dissociate all or one portion of the aggregates. Due to their experimental constraints (implementation time, large amount of samples, lack of sensitivity), these techniques are not really suitable for the characterization of compounds capable of modulating the level of PFβ aggregation.

Techniques based on immunodetection have also been described:
Filtration combined with immuno-detection: in this method the biological sample is filtered on a membrane capable of retaining high molecular weight species and in particular PAFβ. An antibody specific for PAFβ labeled directly or indirectly with a colorimetric or fluorescent or even luminescent tracer is then applied to the membrane. The measured signal is directly proportional to the amount of PAFβ. This method can be adapted to a microplate format to study the aggregation parameters of an amyloid protein or the effect of a compound on its level of aggregation. However, the automation and throughput of the method are limited due to the filtration and washing steps required by this technique [2].
ELISA: different ELISA test strategies have been described in order to allow the characterization of the aggregation of PFβ. The first strategy consists in using the same antibody for the capture of PAFβ on the solid phase and as a tracer allowing their detection. This method allows to detect only PAFβ which have several epitopes of the antibody used in capture and detection. Conversely, the capture antibody and the detection antibody will therefore not be able to be fixed simultaneously on a PNAFβ because it has only one epitope recognized by the antibody used. No signal will therefore be generated in the presence of PNAFβ [3]. The second strategy consists in associating in an ELISA test an antibody specifically recognizing PAFβ with an antibody recognizing all the forms of PFβ present in the sample (PAFβ and PNAFβ) [4]. In this format, the specific antibodies of PAFβ is generally the capture antibody but a format using it as a detection antibody has also been described [5]. A third strategy is to use two different antibodies both recognizing the PAFβ of interest. This last strategy seems to improve the specificity of detection of aggregates [6]. All these ELISA tests allow to determine the level of aggregation of PFβ of interest and to determine the effect of a compound on their level of aggregation. Nevertheless, the detection of PAFβ alone is not enough because, at a given signal, it is not possible to realize the level of aggregation compared to the initial state, unless the measurements are compared to a standard range which necessarily biases the measurements of the tested biological sample.
TR-FRET (Energy transfer in resolved time): kits based on this method have been developed and marketed in order to detect the aggregation of TAU and alpha-synuclein in organic samples (see website Cisbio, commercial references 6FTAUPEG and 6FASYPEG). These tests use the same labeled antibody respectively at the fluorescence donor and acceptor. In the presence of a PAFβ, due to aggregation, the two labeled antibodies will be bonded simultaneously to PAFβ inducing an energy transfer between the donor and the fluorescence acceptor located near each other. The acceptor antibody will then emit a specific FRET signal which will be measured in resolved time. On the other hand, only one of the two antibodies can be bonded on PNAFβ thus preventing any proximity between the donor and the acceptor. It will then be impossible to obtain a FRET signal on PNAFβ. These kits allow to determine the level of aggregation of PFβ of interest and to determine the effect of a compound on its level of aggregation in miniaturized and high speed formats. Nevertheless, these kits require the use of antibodies capable of recognizing epitopes of PFβ even if said epitopes are aggregated, which is sometimes limiting or even blocking in the development of detection tests, since the immunizations are generally made with PNAFβ, which can make it difficult to obtain anti-PAFβ antibodies.

The methods described in the prior art therefore aim at directly detecting a PAFβ in a sample, in particular by using one or more ligands of PAFβ. Nevertheless, directly detecting PAFβ can make these techniques difficult to implement, imprecise and/or little suitable for large scale use. In addition, the only PAFβ detection is not enough because, at a given signal, it is not possible to realize the level of aggregation compared to the initial state, unless to compare the measurements to a standard range which necessarily biases the measurements of the tested biological sample.

An easier, faster and more reliable diagnosis of diseases related to the aggregation of PFβ is therefore necessary.

The Applicant has developed a protocol which is simple and easy to implement which allows to disaggregate all or one portion of the PAFβ present in a sample in order to obtain a PNAFβ. This protocol allowed the Applicant to develop a PAFβ detection method carried out through the measurement of the PNAFβ content, which allows to overcome all the constraints related to the detection of PAFβ.

### SUMMARY OF THE INVENTION

According to a first aspect, the invention relates to an in vitro method for detecting in a sample a β-sheet aggregate form of a protein forming β-sheet aggregates (PAFβ), comprising the following steps:
a) In a first container:
   a1) introducing a sample likely to contain a PAFβ,
   a2) adjusting the pH to a pH ranging from 9.7 to 13.2 to disaggregate all or one portion of the PAFβ in order to obtain a β-sheet non-aggregate form of the protein forming β-sheet aggregates (PNAFβ),
   a3) adjusting the pH to a pH ranging from 6 to 9,
b) Measuring the PNAFβ content in the first container with an appropriate immunological method;
c) In a second container:
   c1) introducing the same sample as in step a1),
   c2) adjusting the pH to a pH ranging from 6 to 9;
d) Measuring the PNAFβ content in the second container using the same method as in step b);
e) Comparing the contents measured in steps b) and d), a decrease in the content measured in step d) compared to the content measured in step b) indicating that the sample contains a PAFβ.

According to a second aspect, the invention relates to an in vitro method for monitoring the therapeutic efficacy of a treatment for a disease associated with PAFβ, comprising the following steps:
A) Implementing the method according to the invention on a first sample in which step e) consists in determining the ratio between the content measured in step b) and the content measured in step d) (“Ratio b)/d) of sample 1”);
B) Implementing the same method as in step A) on a second sample, to determine the “Ratio b)/d) of sample 2”;
C) Comparing the ratios determined in steps A) and B), in which therapeutic efficacy is observed when the ratio determined in step B) is lower than the ratio determined in step A).

According to a third aspect, the invention relates to an in vitro method for measuring the pharmacological efficacy of a molecule on a disease associated with PAFβ, comprising the following steps:
A) Implementing the method according to the invention on a first sample in which step e) consists in determining the ratio between the content measured in step b) and the content measured in step d) (“Ratio b)/d) of sample 1”);
B) Implementing the same method as in step A) on a second sample, to determine the “Ratio b)/d) of sample 2”;
C) Comparing the ratios determined in steps A) and B), in which pharmacological efficacy is observed when the ratio determined in step B) is lower than the ratio determined in step A).

### DETAILED DESCRIPTION

### Definitions

The expression “protein forming β-sheet type aggregates” or “PFβ” designates a protein capable of forming aggregates rich in β sheets, that is to say a protein capable of forming a multimeric form (or an oligomeric form) rich in β sheets. Generally, a non-aggregate form of PFβ is normal, but an aggregate form thereof is characterized, in particular, by a neurodegenerative disease, such as Alzheimer's disease, Creutzfeldt-Jakob disease, Parkinson's disease or amyotrophic lateral sclerosis (ALS). It can be a human or animal, native or recombinant protein. In the context of the present invention, the non-aggregate form of a PFβ is designated by the expression “β-sheet non-aggregate form of a protein forming β-sheet aggregates” or “PNAFβ”. The aggregate form of a PFβ is referred to as “β-sheet aggregate form of a protein forming β-sheet aggregates” or “PAFβ”.

The PFβ, which can be in aggregate form (PAFβ) or in non-aggregate form (PNAFβ), can be selected from FUS (Fused in sarcoma), TAF15, EWSR1, DAZAP1, TIA-1, TTR (transthyretin), cystatin C, β2-microglobulin, R amyloid peptide (such as β 1-40 amyloid peptide or β 1-42 amyloid peptide), TAU (Tubulin-Associated Unit), α-synuclein, β-synuclein, γ-synuclein, Huntingtin (HTT), SOD1 (superoxide dismutase 1), prion, and TDP-43 (TAR DNA-binding protein 43). For example, when PFβ is TDP-43, “TDP-43 NAFβ” will be considered for the non-aggregate form of TDP-43 and of “TDP-43 AFβ” for the aggregate form of TDP-43.

PFβ listed above are in particular implicated in neurodegenerative diseases. For example, FUS implicated in amyotrophic lateral sclerosis, TAF15 implicated in amyotrophic lateral sclerosis, β amyloid peptides implicated in Alzheimer's disease and hereditary cerebral amyloid angiopathy, prion implicated in Creutzfeldt-Jakob disease and spongiform encephalopathy, α-synuclein implicated in Parkinson's disease, TAU protein implicated in Alzheimer's disease, frontotemporal dementias, transthyretin implicated in senile systemic amyloidosis or familial amyloid polyneuropathy, cystatin C implicated in hereditary cerebral amyloid angiopathy, β2-microglobulin implicated in hemodialysis-related amyloidosis, Huntingtin implicated in Huntington's disease, SOD1 implicated in amyotrophic lateral sclerosis, TDP-43 implicated in amyotrophic lateral sclerosis and frontotemporal dementias. Preferably, the PFβ is selected from β 1-42 amyloid peptide, β 1-40 amyloid peptide, α-synuclein or TDP-43.

The sample in which the method of the invention is implemented can be any sample likely to contain at least one PAFβ. It can be a biological sample of human or animal origin, or a sample of cells or tissue cultured in vitro.

The term “in vitro method” means a method implemented outside the human or animal body, for example on microorganisms, organs, tissues, cells, cellular sub-fractions (for example nuclei, mitochondria) or (native or recombinant) proteins. The term “in vitro” encompasses ex vivo.

The sample can for example come from an individual, human or animal, having or being suspected of having a disease associated with a PAFβ, for example a neurodegenerative disease as described above. For example, the sample may be selected from a blood sample, a plasma sample, a serum sample, or a cerebrospinal fluid sample. The sample can also be prepared from tissue or cells from the individual, for example from brain, central nervous system tissue, organs such as spleen and intestine. The sample can therefore be a cell lysate, a cell homogenate, a tissue lysate or a tissue homogenate, such as a brain homogenate. The sample may also comprise cells (for example a cell line), cell sub-fractions (for example nuclei, mitochondria) or (native or recombinant) proteins.

The sample can also come from cells or from a tissue cultured in vitro or ex vivo, preferably from a cell or tissue model of a disease associated with PAFβ. For example, the sample can be selected from cell lysate, cell homogenate, tissue lysate, tissue homogenate, cell culture supernatant, or tissue culture supernatant.

Preferably, the sample is a cell lysate or a cell homogenate.

The term “container” designates a well of a plate, a test tube or any other container suitable for mixing a sample with the reagents necessary for the implementation of the method according to the invention.

Within the meaning of the invention, the term “ligand” denotes a molecule capable of binding to a target molecule. In the context of the invention, the target molecule is PNAFβ. This is then referred to as a “ligand of PNAFβ”. The ligand can be of protein nature (for example a protein or a peptide) or of a nucleotide nature (for example a DNA or an RNA). In the context of the invention, the ligand is advantageously selected from an antibody, an antibody fragment, a peptide or an aptamer, preferably an antibody or an antibody fragment.

Within the meaning of the invention, the term “ligand capable of binding specifically to PNAFβ” or “pair of ligands capable of binding specifically to PNAFβ” designates a ligand or a pair of ligands which binds preferentially to the PNAFβ with respect to the PAFβ, that is to say a ligand or a pair of ligands capable of generating a signal (for example an ELISA signal or a RET signal) with respect to at least one PNAFβ twice higher, for example at least three, at least four, at least five or at least six times higher, than the signal generated with respect to the corresponding PAU. Examples 1-4 and 25 of the present application describe ELISA and FRET methods which allow to easily determine whether a ligand or a pair of ligands is capable of binding specifically to PNAFβ.

Advantageously, the “ligand capable of binding specifically to PNAFβ” or the “pair of ligands capable of binding specifically to PNAFβ” is capable of binding to a PNAFβ with an affinity at least 2 times greater than the affinity for the corresponding PAFβ, for example an affinity at least 2 times higher, at least 3 times higher, at least 4 times higher, at least 5 times higher, at least 6 times higher, at least 7 times higher, at least 8 times higher, at least 9 times higher, at least 10 times higher than the affinity for the corresponding PAFβ. In the case of a pair of ligands capable of binding specifically to PNAFβ, it is not necessary for the two ligands of the pair of ligands to be specific for PNAFβ for the pair of ligands to be specific for PNAFβ. Indeed, it is sufficient for at least one of the two ligands of the pair of ligands to be a ligand specific for PNAFβ for the pair of ligands to be specific for PNAFβ. Nevertheless, both ligands of the pair of ligands may be ligands specific for PNAFβ.

The term “affinity” refers to the strength of all the non-covalent interactions between a ligand and an antigen. Affinity is usually represented by the dissociation constant (Kd). The lower the value Kd, the higher the binding affinity between the ligand and its target. The dissociation constant (Kd) can be measured by well-known methods, for example by FRET, by ELISA or by SPR. The techniques described in the literature therefore make it easy to know whether a ligand or a pair of ligands is specific for PNAFβ.

The term “antibody”, also called “immunoglobulin” refers to a heterotetramer consisting of two heavy chains of approximately 50-70 kDa each (called the H chains for Heavy) and two light chains of approximately 25 kDa each (called the L chains for Light), bound together by intra- and inter-chain disulphide bridges. Each chain is made up, in the N-terminal position, of a variable region or domain, called VL for the light chain, VH for the heavy chain, and in the C-terminal position, of a constant region, made up of a single domain called CL for the light chain and three or four domains called CH1, CH2, CH3, CH4, for the heavy chain. Each variable domain generally comprises 4 “hinge regions” (called FR1, FR2, FR3, FR4) and 3 regions directly responsible for binding with the antigen, called “CDR” (called CDR1, CDR2, CDR3). An “antibody” can be of mammalian (for example human or mouse or rat or camelid), humanized, chimeric, recombinant origin. It is preferably a monoclonal antibody produced recombinantly by genetically modified cells according to techniques widely known to the person skilled in the art. The antibody can be of any isotype, for example IgG, IgM, IgA, IgD or IgE, preferably IgG.

The term “antibody fragment” means any portion of an immunoglobulin obtained by enzymatic digestion or obtained by bio-production comprising at least one disulphide bridge and which is capable of binding to the antigen recognized by the whole antibody, for example Fv, Fab, Fab′, Fab′-SH, F(ab′)2, diabodies, linear antibodies (also called “Single Domain Antibodies” or sdAb, or nanobodies), antibodies with a single chain (for example scFvs). Enzymatic digestion of immunoglobulins with pepsin generates an F(ab′)2 fragment and an Fc fragment split into several peptides. F(ab′)2 is made up of two Fab′ fragments bound by interchain disulphide bridges. The Fab portions are made up of the variable regions and the CH1 and CL domains. The Fab′ fragment is made up of the Fab region and a hinge region. Fab′-SH refers to a Fab′ fragment in which the cysteine residue of the hinge region bears a free thiol group.

The term “tracer” means a chemical or biological agent capable of directly or indirectly emitting a signal which can be detected with an appropriate detection device. It can be a fluorescent, luminescent, radioactive or enzymatic tracer. In a particular embodiment, the tracer is a member of a pair of RET partners.

The term “RET” (from “Resonance Energy Transfer”) designates energy transfer techniques. The RET can be a FRET or a BRET.

The term “FRET” (from “Fluorescence Resonance Energy Transfer”) designates the transfer of energy between two fluorescent molecules. FRET is defined as a non-radiative energy transfer resulting from a dipole-dipole interaction between an energy donor and an energy acceptor. This physical phenomenon requires energy compatibility between these molecules. This means that the emission spectrum of the donor must overlap, at least partially, the absorption spectrum of the acceptor. In accordance with Forster's theory, FRET is a process that depends on the distance separating the two molecules, donor and acceptor: when these molecules are close to each other, a FRET signal will be emitted.

The term “BRET” (from “Bioluminescence Resonance Energy Transfer”) designates the transfer of energy between a bioluminescent molecule and a fluorescent molecule.

The term “pair of RET partners” designates a pair consisting of an energy donor compound (hereinafter “donor compound”) and an energy acceptor compound (hereinafter “acceptor compound”); when in close proximity to each other and when excited at the excitation wavelength of the donor compound, these compounds emit a RET signal. It is known that for two compounds to be RET partners, the emission spectrum of the donor compound must partially overlap the excitation spectrum of the acceptor compound. For example, this is the case of “pairs of FRET partners” when using a fluorescent donor compound and an acceptor compound or of “pair of BRET partners” when using a donor bioluminescent compound and an acceptor compound.

The term “RET signal” designates any measurable signal representative of a RET between a donor compound and an acceptor compound. For example, a FRET signal can therefore be a variation in the intensity or the luminescence lifetime of the donor fluorescent compound or of the acceptor compound when the latter is fluorescent.

«2 Containers» Detection Method

According to a first aspect, the invention relates to an in vitro method for detecting in a sample a β-sheet aggregate form of a protein forming β-sheet aggregates (PAFβ), comprising the following steps:
a) In a first container:
   a1) introducing a sample likely to contain a PAFβ,
   a2) adjusting the pH to a pH ranging from 9.7 to 13.2 to disaggregate all or one portion of the PAFβ in order to obtain a β-sheet non-aggregate form of the protein forming β-sheet aggregates (PNAFβ),
   a3) adjusting the pH to a pH ranging from 6 to 9,
b) Measuring the PNAFβ content in the first container with an appropriate immunological method;
c) In a second container:
   c1) introducing the same sample as in step a1),
   c2) adjusting the pH to a pH ranging from 6 to 9;
d) Measuring the PNAFβ content in the second container using the same method as in step b);
e) Comparing the contents measured in steps b) and d), a decrease in the content measured in step d) compared to the content measured in step b) indicating that the sample contains a PAFβ.

The general principle of the method according to the invention is illustrated in FIG. 1.

Below, the method is described in detail step by step.

Step a)
Step a1) consists in, in a first container, introducing a sample likely to contain a PAFβ. The sample can be prepared beforehand in order to be able to properly carry out the steps of the method, in particular the step of measuring the PNAFβ content. For example, when the sample is a tissue or cells, it can be prepared beforehand by lysing, grinding, filtering and/or dissolving it in an appropriate solvent such as water. The person skilled in the art will have no difficulty in preparing the sample.
Step a2) consists in adjusting the pH to a pH ranging from 9.7 to 13.2 to disaggregate all or one portion of the PAFβ in order to obtain a β-sheet non-aggregate form of the protein forming β-sheet aggregates (PNAFβ). The applicant has indeed noticed that a pH ranging from 9.7 to 13.2 allows to disaggregate PAFβ. This disaggregation phenomenon allows to obtain a PNAFβ from the PAFβ.

This phenomenon of disaggregation at pH ranging from 9.7 to 13.2 is quite surprising since the disaggregation methods described in the prior art rather consist in treating the PNAFβ at acid pH, with powerful detergents and/or or by sonication. For example, the work described in [7] shows different methods for obtaining relatively monomeric amyloid proteins from amyloid fibers. In a first approach, a preparation of amyloid fibers is treated by combining an acid, 88% formic acid, with a strong surfactant type detergent, Sodium Dodecyl Sulfate (SDS). An alternative is to combine a chaotropic agent, a saturated solution of Guanidine Thiocyanate (6.8M) with SDS. In both cases, the authors were able to note the disappearance of amyloid fibers in favor of relatively monomeric amyloid proteins (mixture of monomers and dimers). Another study [8] describes different methods of treating biological samples to disaggregate the amyloid proteins (β 1-42 amyloid peptide) they contain before assaying them via an ELISA test. Extracts of mouse brains or cerebrospinal fluids of patients are treated with a solution of fluorinated alcohol (HFIP) or acid (TFA) coupled with sonication for 15 minutes to disaggregate amyloid proteins. The HFIP or TFA are then removed by drying under a constant flow of nitrogen. The dry samples, containing the disaggregated amyloid proteins, are then taken up in a 1% NH₄OH solution before being analyzed. The authors suggest that these 2 types of treatment allow to disaggregate the 13142 amyloid peptide aggregates and thereby improve their quantification.

Nevertheless, the applicant has shown that the disaggregation methods described in the prior art do not allow to measure the PNAFβ content with immunological methods, which need to be implemented at a physiological pH (cf. Examples 5 to 17).

The method of the invention does not require treatment at an acid pH to disaggregate the PAFβ. On the contrary, the Applicant has not only shown that disaggregation was possible by treating a PAFβ at a pH ranging from 9.7 to 13.2, but also that this treatment was entirely compatible with the subsequent implementation of an immunological method aiming at measuring the PNAFβ content.

Advantageously, step a2) consists in adjusting the pH to a pH ranging from 10 to 13.2, for example a pH ranging from 11 to 13.2, a pH ranging from 11.5 to 13.2, a pH ranging from 12 to 13.2, a pH ranging from 12.5 to 13.2, a pH ranging from 12.8 to 13.2, a pH ranging from 10 to 13, a pH ranging from 11 to 13, a pH ranging from 12 to 13, for example about 12.8.

The pH is adjusted with a base, for example a strong base such as KOH or NaOH, preferably NaOH.

The duration of step a2) may depend on various parameters such as the base used or the PFβ tested. In particular, step a2) may last at least 30 seconds, for example at least 1 minute, at least 2 minutes, at least 5 minutes, at least 10 minutes, at least 15 minutes, for example between 30 seconds and 60 min or between 5 minutes and 30 minutes. The person skilled in the art will have no difficulty in adapting the duration of step a2) to manage to disaggregate all or one portion of the PAFβ in order to obtain a PNAFβ.

Step a3) consists in adjusting the pH to a pH ranging from 6 to 9. This step is important since it allows to implement the immunological method of step b).

Advantageously, step a3) consists in adjusting the pH to a pH ranging from 6.4 to 9, for example a pH ranging from 6.4 to 8.4, a pH ranging from 6 to 8.5, a pH ranging from 7 to 8.5.

The pH is adjusted with an acid, for example a strong acid, such as HCl.

Step b)

Step b) consists in measuring the PNAFβ content in the first container with an appropriate immunological method (or appropriate “immunoassay”).

It is necessary that the immunological method of step b) allows to obtain contents which are comparable with each other. However, it is not necessary for the immunological method in step b) to be quantitative, although it can be. The immunological method of step b) must at least allow the measurement of a relative content that can be compared to another relative content measured by the same immunological method.

In a particular embodiment, the immunological method implemented in step b) uses:
(i) a ligand capable of binding specifically to PNAFβ, said ligand being labeled with a tracer, or
(ii) a pair of ligands capable of binding specifically to PNAFβ, at least one ligand of said pair of ligands being labeled with a tracer.

The ligand (i) can be selected from an antibody, an antibody fragment, a peptide or an aptamer, preferably an antibody. The pair of ligands (ii) can be selected from a pair of antibodies, a pair of antibody fragments, a pair of peptides or a pair of aptamers, preferably a pair of antibodies.

The person skilled in the art will have no difficulty in obtaining and/or selecting antibodies or antibody fragments having the desired properties, for example by immunizing a mouse with a PFβ, by carrying out a lymphocyte hybridization from the lymphocytes of the spleen of the immunized mouse in order to generate hybridomas and by testing the antibodies of each hybridoma for their capacities to bind specifically to PNAFβ. Examples of a complete protocol for the generation of anti-PNAFβ antibodies then for the selection of specific antibodies are detailed in examples 1-4 and 25. It is therefore very easy to obtain antibodies or pairs of antibodies directed against any PNAFβ for the implementation of the method according to the invention, for example by implementing the methods described in Examples 1-4 and 25.

Obtaining PNAFβ and PAFβ is within the reach of the person skilled in the art. For example, for proteins called “prion-like” proteins, such as TDP-43, FUS, TAF15 and EWSR1, the production of these proteins fused to Gluthatione-S-Transferase (GST) (N- or C-terminal fusion depending on proteins) allows to obtain GST fusion proteins whose turbidimetric analysis shows that they are in non-aggregate form [9][10][13]. When a Tobacco etch Virus protease (TEV) cleavage site is inserted between the PFβ of interest and the GST, a treatment of the GST proteins with TEV followed by a purification step allows to obtain the GST proteins without label. When said proteins are left for 1 h 30 at room temperature with stirring, turbidimetry analysis shows that they are in aggregate form [10][13]. PFβ fused to GST are also commercially available, for example from Abnova: GST-TDP-43 (Ref. H00023435-P02), GST-FUS1 (Ref. H00002521-P01), GST-TAF15 (Ref. H00008148-P02) GST-EWSR1 (Ref. H00002130-Q01).

Amyloid peptides, in particular the β 1-40 and β 1-42 forms, are also available from many suppliers in powder form. The solubilization of these powders in HFIP or NH₄OH allows to obtain stock solutions containing more than 90% of non-aggregate forms. The extemporaneous use of these solutions previously diluted in buffers with physiological pH values allows to have samples of non-aggregate forms. Conversely, an incubation of several hours to several days, for example an incubation of more than 24 hours, of these same stock solutions in a physiological buffer at room temperature allows to obtain a sample containing very high proportions of aggregate forms [11].

Samples containing PNAFβ and PAFβ can also be obtained from the company StressMarq (www.stressmarq.com). This is particularly the case for alpha, beta and gamma synucleins, the TAU protein, the Cu/Zn superoxide dismutase 1 (SOD1) protein or Transthyretin (TTR).

Advantageously, the immunological method implemented in steps b) is an ELISA method or a RET method, such as a FRET or a BRET.

Depending on the method used, the measurement of step b) can be done directly in the first container by adding the appropriate reagents or in another container with all or one portion of the contents of the first container. For example, reagents for a RET method can be added directly to the first container. Conversely, the ELISA method will preferably be carried out in another container adapted to the implementation of the ELISA method, in particular in a container at the bottom of which a PNAFβ ligand has previously been immobilized.
In a particular embodiment, step b) is carried out by a RET method and consists in:
(b1) introducing into the container a first PNAFβ ligand labeled with a first member of a pair of RET partners and a second PNAFβ ligand labeled with a second member of the pair of RET partners, the pair of ligands being able to bind specifically to PNAFβ, and
(b2) measuring the RET signal emitted in the container.

Obviously, for the implementation of the RET method, the first ligand and the second ligand must not compete for binding to PNAFβ, for example the first ligand and the second ligand must not bind the same epitope on PNAFβ. It is easy to select an appropriate pair of ligands by carrying out the method described in examples 1-4 and 25.

The ligands can be labeled directly or indirectly. The direct labeling of the ligand by a member of a pair of RET partners can be carried out by conventional methods known to the person skilled in the art, based on the presence of reactive groups on the ligand. For example, when the ligand is an antibody or an antibody fragment, the following reactive groups can be used: the terminal amino group, the carboxylate groups of aspartic and glutamic acids, the amine groups of lysines, the guanidine groups of arginines, the thiol groups of cysteines, the phenol groups of tyrosines, the indole rings of tryptophans, the thioether groups of methionines, the imidazole groups of histidines.

The reactive groups can form a covalent bond with a reactive group carried by a member of a pair of RET partners. The appropriate reactive groups, carried by the member of a pair of RET partners, are well known to the person skilled in the art, for example a donor compound or an acceptor compound functionalized by a maleimide group will for example be capable of binding covalently with the thiol groups carried by the cysteines carried by a protein or a peptide, for example an antibody or an antibody fragment. Similarly, a donor/acceptor compound carrying an N-hydroxysuccinimide ester will be able to bind covalently to an amine present on a protein or a peptide.

The ligand can also be labeled with a fluorescent or bioluminescent compound indirectly, for example by introducing an antibody or antibody fragment into the measurement medium, itself covalently bound to an acceptor/donor compound, this second antibody or antibody fragment specifically recognizing the ligand.

Another very conventional means of indirect labeling consists in attaching biotin to the ligand to be labeled, then incubating this biotinylated ligand in the presence of streptavidin labeled with an acceptor/donor compound. Suitable biotinylated ligands can be prepared by techniques well known to the person skilled in the art; Cisbio Bioassays, for example, markets streptavidin labeled with a fluorophore, the trade name of which is “d2” (ref. 610SADLA).

Advantageously, one of the members of the pair of RET partners is a fluorescent donor or luminescent donor compound and the other member of the pair of RET partners is a fluorescent acceptor compound or a non-fluorescent acceptor compound (quencher).

When the RET is a FRET, the donor fluorescent compound can be a FRET partner selected from: a europium cryptate, a europium chelate, a terbium chelate, a terbium cryptate, a ruthenium chelate, a quantum dot, allophycocyanins, rhodamines, cyanines, squaraines, coumarins, proflavins, acridines, fluoresceins, boron-dipyrromethene derivatives and nitrobenzoxadiazole. When the RET is a FRET, the acceptor fluorescent compound can be a FRET partner selected from: allophycocyanins, rhodamines, cyanines, squaraines, coumarins, proflavins, acridines, fluoresceins, boron-dipyrromethene derivatives, nitrobenzoxadiazole, a quantum dot, GFP, GFP variants selected from GFP10, GFP2 and eGFP, YFP, YFP variants selected from eYFP, YFP topaz, YFP citrine, YFP venus and YPet, mOrange, DsRed.

When the RET is a BRET, the donor luminescent compound can be a partner of BRET selected from: Luciferase (luc), Renilla Luciferase (Rluc), variants of Renilla Luciferase (Rluc8) and Firefly Luciferase. When the RET is a BRET, the acceptor fluorescent compound is a BRET partner selected from: allophycocyanins, rhodamines, cyanines, squaraines, coumarins, proflavins, acridines, fluoresceins, boron-dipyrromethene derivatives, nitrobenzoxadiazole, a quantum dot, GFP, GFP variants selected from GFP10, GFP2 and eGFP, YFP, YFP variants selected from eYFP, YFP topaz, YFP citrine, YFP venus and YPet, mOrange, DsRed.

In a particular embodiment, step b) is carried out by an ELISA method and consists in:
(b1) introducing all or one portion of the sample into a container at the bottom of which a first PNAFβ ligand has been immobilized, then introducing a second PNAFβ ligand labeled with a tracer, the pair of ligands being capable of binding specifically to the PNAFβ,
(b2) measuring the ELISA signal emitted in the container.

The ELISA method is widely described in the prior art and does not have any difficulty of implementation for the person skilled in the art.

Step c)

Step c) consists in, in a first container, c1) introducing the same sample as in step a1).

Contrary to step a), the pH is not adjusted to a pH ranging from 9.7 to 13.2 during step c). The pH is directly adjusted to a pH ranging from 6 to 9 (step c2)), preferably at the same pH as the pH of step a3).

The pH in step c2) can be adjusted with an acid/base mixture, for example a strong acid/strong base mixture. It may be, for example, an NaOH/HCl mixture. Preferably, the acid used in step c2) is the same as that used in step a3) and the base used in step c2) is the same as the base used in step a2).

Step d)

Step d) consists in measuring the PNAFβ content in the second container with the same method as in step b). Step d) is implemented in the same way as in step b) to be able to compare the measurements and implement step e).

Thus, when step b) is carried out by a RET method, step d) consists in:
(d1) introducing into the container a first PNAFβ ligand labeled with a first member of a pair of RET partners and a second PNAFβ ligand labeled with a second member of the pair of RET partners, the pair of ligands being able to bind specifically to PNAFβ, and
(d2) measuring the RET signal emitted in the container.

In the same way, when step b) is carried out by an ELISA method, step d) consists in:
(d1) introducing all or one portion of the sample into a container at the bottom of which a first PNAFβ ligand has been immobilized, then introducing a second PNAFβ ligand labeled with a tracer, the pair of ligands being capable of binding specifically to the PNAFβ,
(d2) measuring the ELISA signal emitted in the container.

Step e)

Step e) consists in comparing the contents measured in step b) and in step d), a decrease in the content measured in step d) compared to the content measured in step b) indicating that the sample contains a PAFβ.

Obviously, if the first and the second container are swapped, step e) will consist in comparing the contents measured in step b) and in step d), an increase in the content measured in step d) relative to the content measured in step b) indicating that the sample contains a PAFβ.

The person skilled in the art can easily compare the contents measured in steps b) and d) and define a threshold enabling him to qualify the increase or decrease. For example, a difference between the measured contents greater than 5%, greater than 10%, greater than 15%, greater than 20%, greater than 25%. The determination of the threshold may depend on the variability inherent in the immunological method chosen.

The person skilled in the art could, for example, calculate the ratio between the contents measured in steps b) and d). In general, the greater the difference between the measured contents, the greater the ratio between the measured contents and the greater the amount of PAFβ in the sample.

When the immunological method is an ELISA method or a RET method, the RET signals or the ELISA signals measured in steps b) and d) will be compared. Therefore, a decrease in RET signal or a decrease in ELISA signal will indicate that the sample contains PAFβ. Advantageously, a ratio will be calculated between the signal measured in the first container (step b)) and the signal measured in the second container (step d)). The calculation of the ratio can be done manually or automatically.

Preferably, the duration between step a3) and step b) and/or between step c2) and step d) will be adapted to prevent the PNAFβ from re-aggregating into PAFβ. The duration will preferably be less than 24 hours, for example less than 5 hours. Advantageously, steps b) and d) are carried out following steps a3) and c2) respectively, that is to say less than 30 minutes after steps a3) and c2). The person skilled in the art will have no difficulty in adapting the duration of step a3) and/or of step c2) to prevent all or one portion of the PNAFβ from re-aggregating into PAFβ before step c) and/or step d).

The comparison of the contents in the two samples in step e) allows to measure the level of aggregation very precisely, in particular compared to most methods of the prior art.

Method for Monitoring Therapeutic Efficacy

The present invention also relates to an in vitro method for monitoring the therapeutic efficacy of a treatment for a disease associated with PAFβ in a patient, comprising the following steps:
A) Implementing the method according to the invention on a first sample of said patient, in which step e) consists in determining the ratio between the content measured in step b) and the content measured in step d) (“Ratio b)/d) of sample 1”);
B) Implementing the same method as in step A) on a second sample of said patient, to determine the “Ratio b)/d) of sample 2”;
C) Comparing the ratios determined in steps A) and B), in which therapeutic efficacy is observed when the ratio determined in step B) is lower than the ratio determined in step A).

The treatment may be a known treatment for the disease associated with PAFβ or an experimental treatment. The sample is preferably from an individual with PAFβ-associated disease.

To be able to monitor the effectiveness of a treatment, the first sample and the second sample are taken from the patient at different times, for example the first sample is taken at a time T1 and the second sample is taken at a time T2. Advantageously, the first sample is taken before the second sample, for example the first sample is taken at a time T1 before the treatment of the patient or during said treatment, and the second sample is taken at a time T2 after said treatment or during said treatment. The time that elapses between the collection of the first sample and the collection of the second sample will be chosen in order to be able to detect the therapeutic effectiveness of the treatment.

Method for Monitoring the Measurement of the Pharmacological Efficacy

The present invention also relates to an in vitro method for measuring the pharmacological efficacy of a drug molecule or a drug candidate on a disease associated with PAFβ in a test sample, comprising the following steps:
A) Implementing the method according to the invention on a first sample of said test sample, in which step e) consists in determining the ratio between the content measured in step b) and the content measured in step d) (“Ratio b)/d) of sample 1”);
B) Implementing the same method as in step A) on a second sample of said test sample, to determine the “Ratio b)/d) of sample 2”;
C) Comparing the ratios determined in steps A) and B), in which pharmacological efficacy is observed when the ratio determined in step B) is lower than the ratio determined in step A).

The molecule can be a drug or a drug candidate. The sample preferably comes from cells or tissue cultured in vitro. Consequently, the drug molecule or the drug candidate is preferably tested on an in vitro model of disease associated with a PAFβ. Such models are widely described in the literature.

The test sample corresponds to a sample which allows to test a drug or a drug candidate in vitro, it may be for example a sample of cells cultured in vitro or a sample of tissue cultured in vitro. The test sample corresponds to what is commonly called an “in vitro model of disease associated with PAFβ”. Thus, in order to be able to measure the pharmacological efficacy of a drug molecule or a drug candidate, the first sample and the second sample are taken from the test sample at different times, for example the first sample is taken at a time T1 and the second sample is taken at a time T2. Advantageously, the first sample is taken before the second sample, for example the first sample is taken at a time T1 before the treatment of the test sample with a drug molecule or a drug candidate, and the second sample is taken at a time T1 after said treatment. The time that elapses between taking the first sample and taking the second sample will be chosen in order to be able to detect pharmacological efficacy of the drug molecule or the drug candidate.

The present invention will now be illustrated by the following non-limiting examples.

### EXAMPLES

### Example 1: Method for Identifying Antibodies Specific to a PNAFβ

Generating Anti-PNAFβ Antibodies

Mice Immunizations

BALB/c mice are immunized by injection of the PNAFβ protein previously diluted in phosphate buffer prepared under physiological conditions. The absence of the presence of PNAFβ multimers or aggregates is virified in the buffer intended for the injections in order to direct the immune response of the mice to the non-aggregate forms. The first injection is followed by three boosters at monthly intervals.

Fifteen days after each injection, blood punctures are carried out on the mice to verify the presence of an immune response by titration of the antibodies.

Titration of Immune Sera in Anti-PNAFβ Antibodies by ELISA Tests

For this purpose, ELISA-type immunodetection tests are implemented depending on the nature of the PFβ. For amyloid-type PFβs or peptides, PNAFβ is previously labeled on its primary lysines with biotin using a reagent composed of biotin, a carbon linker and an NHS (N-Hydroxysuccinimide) reactive group. For non-amyloid or amyloid PFβ, PNAFβ in GST fusion is directly immobilized on 96-well plates via the GST tag by using ELISA microplates functionalized with a glutahion group. Biotin-labeled proteins are immobilized on 96-well ELISA plates via biotin using microplates functionalized with streptavidin. For this purpose, 100 μl of GST fusion and/or biotinylated PNAFβ solution are added to each well and then incubated for 2 h at room temperature. The wells are then washed three times in PBS buffer supplemented with 0.05% Tween-20. After removing the washing solution, each well is then incubated overnight at 4° C. with 200 μL of a blocking solution composed of PBS, 5% BSA.

The serial dilutions by a factor of 10 to 100 million of the blood samples (immune sera) are then added, in doublets, at a level of 100 μL per well in PBS+0.1% BSA buffer and incubated with stirring for 2 hours. The non-specific antibodies not bound to the immobilized PNAFβ are eliminated by three washing steps of 200 μl in PBS 1× buffer, 0.05% Tween20. The possible presence of specific antibodies is detected using 100 μL per well of mouse anti-Fc secondary antibody coupled to HRP (horseradish peroxidase) (Sigma #A0168 diluted to 1/10000 in PBS, BSA 0.1%). After 1 hour of incubation at room temperature with stirring then three washes under 200 μL in PBS 1× buffer, 0.05% Tween20, the revelation of the HRP is carried out by colorimetric assay at 450 nm following incubation of its TMB substrate (3,3′,5,5′-Tetramethylbenzidine, Sigma #T0440) for 20 min at room temperature and with stirring. This blocking solution is then removed by aspiration and the plates are stored at 4° C. for future use.

In order to ensure that the antibodies detected by the ELISA test are indeed directed against the PNAFβ protein and not against the GST tag or biotin, the same punctures are tested on the ELISA test after pre-incubation with an excess of another orthogonal protein tagged with GST or biotin. Thus, the anti-TAG antibodies bind to the tagged orthogonal protein and therefore not to the PNAFβ protein immobilized at the bottom of the wells; in which case no HRP signal or a decrease in HRP signal is detected.

Fusion & Cloning

The mice having the best anti-PNAFβ antibody titers (signal, that is to say high optical density at 450 nm) and the least drop in signal in the anti-TAG control case are selected for the next step of lymphocyte hybridization, also called fusion. The mouse spleen is extracted to isolate a mixture of lymphocytes and plasma cells. This multi-cell sample is fused in vitro with a myeloma cell line in the presence of a cell fusion catalyst of the polyethylene glycol (PEG) type. A mutant myeloma cell line, deficient for the enzyme HGPRT (Hypoxanthine Guanosin Phosphoribosyl Transferase) is used to allow selection of hybrid cells, called hybridomas. These cells are cultured in a medium containing hypoxanthine, aminopterin (methotrexate) and thyamine (HAT medium), to allow the elimination of unfused myeloma cells and thus select the hybridomas of interest. Unfused spleen cells, on the other hand, die since they are unable to proliferate in vitro. Thus, only hybridomas survive this selection pressure in vitro.

These hybridomas are cultured in culture plates. The supernatants of these hybridomas are tested to assess their ability to produce anti-PNAFβ antibodies. For this purpose, an ELISA test as described above is carried out. The minimum threshold used to select a clone is four times that of the non-specific. The best hybridomas are cloned with a limiting dilution step in order to obtain stable hybridoma clones.

The clones selected are cultured with a view to forming a bank of hybridomas, tested for cell viability and stored in liquid nitrogen. At this step, the antibodies produced by the clones can be easily sequenced by methods well described in the prior art in order to be able to produce the antibodies, for example, in producer cells. Alternatively, antibodies are produced as described below.

Production of Anti-PNAFβ Antibodies

The clones of hybridomas of interest are returned to culture and the cellular inoculum is then injected into BALB/c mice (intraperitoneal injection, IP) in order to allow the production of antibodies in large amounts in the liquid of ascites.

After characterization of the content of ascites fluids by various techniques aiming at quantifying and qualifying the antibody content, said antibodies are then purified after optional precipitation with salts, via affinity chromatography on columns including resins grafted with protein A. After washing the column to remove the constituents apart from the antibodies, the antibody content is eluted by shocking at acid pH in glycine buffer. After pH neutralization and dialysis against a buffer at neutral pH, the anti-PNAFβ antibodies are ready for storage at 4° C. or freezing and subsequent use/characterization (isotyping, assay, functional tests).

Selection of a Pair of Antibodies Capable of Binding Specifically to a PNAFβ (ELISA Method)

In order to select a pair of antibodies preferentially recognizing a PNAFβ vis-à-vis a PAFβ, an ELISA type test is implemented. For this purpose, one of the antibodies of the tested pair is biotinylated using the Lightning-Link Rapid Biotin Type A kit (Expedeon, reference SKU 370-0005) according to the supplier's recommendations. The second antibody of the tested pair, diluted beforehand in PBS buffer at concentrations comprised between 1 and 20 μg/mL, is adsorbed onto 96-well plates of the “high binding” ELISA type. For this purpose, 100 μL of antibodies are added to each well then incubated for 20 hours at 4° C. followed by three washes in PBS 1× buffer, 0.05% Tween20. After removing the washing solution, each well is then incubated overnight at 4° C. with 200 μL of a blocking solution composed of PBS, 5% BSA. This blocking solution is then removed by aspiration and the plates are stored at 4° C. for future use.

Serial dilutions by a factor of 1 to 1/100th of samples containing the same initial concentration of PNAFβ or of PAFβ are added at a level of 100 μL/well and incubated for 2 hours at room temperature with stirring. The PNAFβ or PAFβ not bound to the antibody adsorbed on the plates are eliminated by three washing steps in PBS 1× buffer, 0.05% Tween20. The biotinylated antibody, previously diluted in PBS buffer to a concentration comprised between 10 and 200 ng/mL, is added at a level of 100 μL/well and incubated for 2 hours at room temperature with stirring. The biotinylated antibodies not bound to PNAFβ or PAFβ are eliminated by three washing steps in PBS 1× buffer, 0.05% Tween20. The detection of bound antibodies is carried out using a streptavidin-HRP (R&D Systems, Ref. DY998) diluted to 1/10th in PBS, 0.1% BSA. After 30 minutes of incubation at room temperature with stirring, then three washes in PBS 1× buffer, 0.05% Tween20, the revelation of the HRP is carried out by measuring the optical density at 450 nm (O.D. 450 nm) following the incubation of its TMB substrate (3,3′,5,5′-Tetramethylbenzidine, Sigma #T0440) for 20 min at room temperature with stirring.

In a first step, the reference dilution of the samples of PNAFβ or PAFβ is determined by analyzing the O.D. 450 nm measured with the different dilutions of PNAFβ. As indicated in FIG. 2, the reference dilution is that for which 80% of the maximum O.D. 450 nm is obtained.

In a second step, the O.D. 450 nm obtained with the reference dilution of the sample of PNAFβ is compared with the O.D. 450 nm measured with an identical dilution of the sample of PAFβ. As shown in FIG. 3, a pair of antibodies able to bind specifically to PNAFβ will give a 50% lower O.D. 450 nm on the PAFβ sample compared to the O.D. 450 nm measured on the PNAFβ sample.

Selection of a Pair of Antibodies Capable of Binding Specifically to a PNAFβ (FRET Method)

In order to select a pair of antibodies preferentially recognizing a PNAFβ over a PAFβ, a FRET test is set up. This test is based on Cisbio Bioassays HTRF® Technology. The principle of this technique is based on a fluorescence energy transfer between a donor molecule, a Terbium cryptate (Donor), and a fluorescent energy acceptor molecule d2 (Acceptor). These two fluorescent molecules are grafted by covalent coupling to antibodies to implement an immunological assay as illustrated in FIG. 4. For this purpose, one of the antibodies of the tested pair is labeled with the donor Lumi4 Terbium using the kit Terbium Cryptate labeling kit (Cisbio Bioassays, reference 62TBSPEA) according to the manufacturer's recommendations. Before use, the donor-labeled antibody is diluted in a 20 mM Hepes buffer pH=7.4, 0.1% BSA at a concentration of 0.5 nM. The second antibody of the tested pair is labeled with the acceptor d2 using the d2 labeling kit (Cisbio Bioassays reference 62D2DPEA) according to the supplier's recommendations. Before use, the donor-labeled antibody is diluted in a 20 mM Hepes buffer pH=7.4, 0.1% BSA at a concentration of 5 nM. Serial dilutions of a factor of 1 to 1/100th of samples containing the same initial concentration of PNAFβ or PAFβ are distributed in a 384 microplate at a level of 16 μL/well. 2 μL of the 0.5 nM solution of donor-labeled antibody and 2 μL of the 5 nM solution of acceptor-labeled antibody are then added to each well. The microplate is incubated for 20 h at room temperature. The detection of the FRET signal in the different plates was carried out on a PHERAstar FS Lamp apparatus (BMG Labtech) using an HTRF detection module.

In a first step, the reference dilution of the samples of PNAFβ or PAFβ is determined by analyzing the HTRF signal measured with the different dilutions of PNAFβ. As indicated in FIG. 5, the reference dilution is that for which 80% of the maximum HTRF signal is obtained, before the saturation plateau of the immunoassay.

In a second step, the HTRF signal obtained with the reference dilution of the sample of PNAFβ is compared with the HTRF signal measured with an identical dilution of the sample of PAFβ. As illustrated in FIG. 6, a pair of antibodies capable of binding specifically to PNAFβ will give a 50% lower HTRF signal on the PAFβ sample compared to the HTRF signal measured on the PNAFβ sample.

### Example 2: Method Allowing to Identify Pairs of Antibodies Capable of Binding Specifically to TDP-43 NAFβ (FRET Method)

An alternative method for obtaining samples containing TDP-43 AFβ or TDP-43 NAFβ consists in culturing HeLa cells and treating them or not for at least 6 hours with staurosporine. Analysis of cell lysates by SDS-PAGE and Western-Blot shows that untreated HeLa cell lysates contain TDP-43 NAFβ while HeLa lysates treated with staurosporine essentially contain TDP-43 AFβ [12].

The method is based on the FRET technology (HTRF® technology from Cisbio Bioassays) described in example 1.

Preparation of the Antibodies to be Tested

Five antibodies directed against TDP-43 were labeled respectively with the Donor and with the Acceptor. These labelings were carried out using the labeling kits marketed by Cisbio Bioassays (Commercial references 62EUSUEA and 62D2DPEA). The labeling rates obtained (number of fluorescent molecules per antibody) were in line with expectations. Donor marking rates were comprised between 5.9 and 7.9. Acceptor labeling rates were comprised between 2.3 and 3.3.

The table below gives the characteristics of the antibodies which were tested.

TABLE 1

| Identification of the | Commercial | |
|---|---|---|
| antibody | reference | Supplier |
| Ac1 | SIG-39854 | Biolegend |
| Ac2 | 89789BF | Cell Signaling Technology |
| Ac3 | MABN774 | Merck Millipore |
| Ac4 | ab238443 | Abcam |
| Ac5 | ab248546 | Abcam |

To be tested, all the antibodies were diluted in a buffer to obtain respective concentrations of 3 nM for the antibodies labeled with the Donor and 30 nM for those labeled with the Acceptor.

Preparation of a Sample of TDP-43 NAFβ (Monomers)

HeLa cells were seeded with 5 million cells in a flask (175 cm²) in complete culture medium (MEM alpha medium+2 mM Hepes+10% decomplemented fetal calf serum+1% antibiotics, Penicillin 5000 U/ml and Streptomycin 5000 μg/ml). After 78 hours, the culture medium was aspirated. The cells were then lysed with a cell lysis buffer. The lysate obtained (hereinafter “Monomer sample”), containing the TDP-43 NAFβ, was frozen at −80° C. with a view to its use. This sample was checked by western blot as recommended in the literature [12] in order to ensure that it essentially contains non-aggregated TDP43 (TDP-43 NAFβ).

Preparation of a Sample of TDP-43 AFβ (Aggregates)

HeLa cells were seeded with 5 million cells in a flask (175 cm²) in complete culture medium (MEM alpha medium+2 mM Hepes+10% decomplemented fetal calf serum+1% antibiotics, Penicillin 5000 U/ml and Streptomycin 5000 μg/ml). After 72 hours, the culture medium was aspirated. A staurosporine solution at 1 μM in complete medium was added to the cells in culture for 6 h. Treatment of cells with staurosporine allows TDP-43 to be aggregated to obtain TDP-43 AFβ. The culture medium was then aspirated before adding cell lysis buffer. The lysate obtained (hereinafter “Aggregate sample”), containing the TDP-43 AM, was frozen at −80° C. with a view to its subsequent use. This sample was checked by western blot as recommended in the literature [12] to ensure that it essentially contains aggregated TDP43 (TDP-43 AFβ).

Screening of Pairs of Antibodies on Monomers or Aggregates

On the day of the test, the Monomer and Aggregate samples were thawed before distribution in 384-well microplates (Greiner reference 784075).

The following reagents were added to the microplates in the order below:
16 μL of Monomer sample or Aggregate sample,
2 μL of Donor antibody
2 μL of Acceptor antibody

The plates were then incubated overnight at room temperature.

The detection of the FRET signal in the various plates was carried out on a PHERAstar FS Lamp apparatus (BMG Labtech) using an HTRF detection module. For all the pairs of tested antibodies, the signals obtained in FRET on the Monomer sample and on the Aggregate sample were compared by calculating a ratio of the FRET signals (Monomers/Aggregates), as shown in FIG. 7 with the pair Ac1-Donor/Ac4-Acceptor.

The ratio of FRET signals (Monomers/Aggregates) was calculated for all the pairs of tested antibodies. Table 2 below summarizes the results obtained. All pairs of antibodies with a ratio (Monomer/Aggregates) greater than 2 are considered selective for TDP-43 NAFβ with respect to TDP-43 AFβ.

TABLE 2

Donor

| | | Ac1 | Ac2 | Ac3 | Ac4 | Ac5 |
|---|---|---|---|---|---|---|
| Acceptor | Ac1 | | 5.5 | 1.0 | 1.1 | 4.2 |
| | Ac2 | 2.2 | | 1.1 | 1.1 | 1.9 |
| | Ac3 | 1.1 | 0.9 | | 1.1 | 1.0 |
| | Ac4 | 6.0 | 1.3 | 1.0 | | 2.9 |
| | Ac5 | 3.9 | 3.0 | 1.0 | 1.1 | |

7 pairs of antibodies allowed to discriminate between TDP-43 NAFβ and TDP-43 AFβ because they have a Monomer/Aggregate ratio greater than 2. They are listed in Table 3.

TABLE 3

| Donor | Acceptor |
|---|---|
| Ac1 | Ac2 |
| Ac1 | Ac4 |
| Ac1 | Ac5 |
| Ac2 | Ac1 |
| Ac2 | Ac5 |
| Ac5 | Ac1 |
| Ac5 | Ac4 |

### Example 3: Method for Identifying Pairs of Antibodies Capable of Binding Specifically to the Beta 1-40 Amyloid Peptide NAFβ (FRET Method)

The method is based on FRET technology (HTRF® technology from Cisbio Bioassays), as detailed in Example 1.

Pair of Tested Antibodies

The antibodies directed against the beta 1-40 amyloid peptide labeled respectively with the Donor and with the Acceptor are those contained in the Amyloid Beta 1-40 HTRF kit marketed by Cisbio Bioassays (reference 62B40PEG). They were diluted in the reference diluent 62RB3FDG as recommended by the Amyloid Beta 1-40 HTRF kit manual.

Preparation of Beta 1-40 Amyloid Peptide NAFβ (Monomers)

The freeze-dried human beta 1-40 amyloid peptide (ERI275BAS, The ERI Amyloid Laboratory, LLC, Oxford) was resuspended according to the supplier's recommendations then diluted in 10 mM Sodium Phosphate pH 7.4 buffer to a concentration of 30 μM. The solution (hereinafter “Monomer sample”), containing the beta 1-40 amyloid peptide NAFβ, was then frozen at −80° C. A Thioflavin T test was performed on this solution. It indicates that it contains more than 90% beta 1-40 amyloid peptide monomers.

Preparation of Beta 1-40 Amyloid Peptide AFβ (Aggregates)

The freeze-dried human beta 1-40 amyloid peptide (ERI275BAS, The ERI Amyloid Laboratory, LLC, Oxford) was resuspended according to the supplier's recommendations then diluted in 10 mM Sodium Phosphate pH 7.4 buffer to a concentration of 30 μM. This solution is then incubated for 495 hours at 25° C., which allows to aggregate the beta 1-40 amyloid peptide to obtain the beta 1-40 amyloid peptide AFβ. The solution (hereinafter “Aggregate sample”), containing the beta 1-40 amyloid peptide AFβ, was then frozen at −80° C. A Thioflavin T test was performed on this solution. It indicates that it contains more than 90% beta 1-40 amyloid peptide aggregates.

Test of the Pair of Antibodies on Monomers or Aggregates

On the day of the test, the Monomer and Aggregate samples were thawed then diluted in 10 mM Sodium Phosphate pH 7.4 buffer at a concentration of 20.3 ng/mL before distribution in a 384-well microplate (Greiner reference 784075).

The following reagents were added to the microplates in the order below:
5 μL of Monomer sample or Aggregate sample
5 μL of diluent (Ref. 62DL1DDD, Cisbio Bioassays)
5 μL of donor antibody
5 μL of acceptor antibody

The plates were then incubated overnight at a temperature comprised between 2° C. and 8° C.

The detection of the FRET signal in the different plates was carried out on a PHERAstar FS Lamp apparatus (BMG Labtech) using an HTRF detection module. The signals obtained in FRET on the Monomer sample and on the Aggregate sample were compared by calculating the ratio of FRET signals (Monomers/Aggregates) as shown in FIG. 8.

The ratio of the FRET (Monomer/Aggregate) signals of the pair of antibodies is greater than 2 (value of 3.1). This indicates that this pair of antibodies discriminates beta 1-40 amyloid peptide NAFβ from beta amyloid peptide 1-40 AFβ, that is to say this pair of antibodies is specific for beta amyloid peptide 1-40 NAFβ.

### Example 4: Method for Identifying Pairs of Antibodies Capable of Binding Specifically to the Beta Amyloid Peptide 1-42 NAFβ (FRET Method)

The method is based on FRET technology (HTRF® technology from Cisbio Bioassays), as detailed in Example 1.

Pair of Tested Antibodies

Two antibodies directed against the beta amyloid peptide 1-42 labeled respectively with the Donor and the Acceptor were diluted to respective concentrations of 3 nM (Donor) and 30 nM (Acceptor).

Preparation of Beta 1-42 Amyloid Peptide NAFβ (Monomers)

The freeze-dried human beta amyloid peptide 1-42 (The ERI Amyloid Laboratory, LLC, Oxford) was resuspended according to the supplier's recommendations then diluted in 10 mM Sodium Phosphate pH 7.4 buffer to a concentration of 30 μM. The solution (hereinafter “Monomer sample”), containing the beta amyloid peptide 1-42 NAFβ, was then frozen at −80° C. A Thioflavin T test was performed on this solution. It indicates that it contains more than 90% beta amyloid peptide 1-42 monomers.

Preparation of Beta Amyloid Peptide 1-42 AFβ (Aggregates)

The freeze-dried human beta amyloid peptide 1-42 (ERI275BAS, The ERI Amyloid Laboratory, LLC, Oxford) was resuspended according to the supplier's recommendations then diluted in 10 mM Sodium Phosphate pH 7.4 buffer to a concentration of 30 μM. This solution is then incubated for 188 hours at 25° C. The solution (hereinafter “Aggregate sample”), containing the beta 1-42 amyloid peptide AFβ, was then frozen at −80° C. A Thioflavin T test was performed on this solution. It indicates that it contains more than 90% beta amyloid peptide 1-42 aggregates.

Test of the Pair of Antibodies on Monomers or Aggregates

On the day of the test, the Monomer and Aggregate samples were thawed then diluted in 10 mM Sodium Phosphate pH 7.4 buffer at a concentration of 2.4 ng/mL before distribution in 384-well microplates (Greiner reference 784075).

The following reagents were added to the microplates in the order below:
16 μL of Monomer sample or Aggregate sample
2 μL of Donor antibody
2 μL of Acceptor antibody

The plates were incubated overnight at a temperature comprised between 2° C. and 8° C.

The detection of the FRET signal in the various plates was carried out on a PHERAstar FS Lamp apparatus (BMG Labtech) using an HTRF detection module. The signal obtained in FRET on the Monomer sample and on the Aggregate sample were compared by calculating the ratio of FRET signals (Monomer/Aggregates) as shown in FIG. 9.

The ratio of the FRET signals (Monomer/Aggregate) of the pair of antibodies studied is greater than 2 (value of 4.5). This indicates that this pair of antibodies discriminates beta 1-42 amyloid NAFβ from beta amyloid 1-42 AFβ, that is to say this pair of antibodies is specific for the beta amyloid peptide 1-42 NAFβ.

### Example 5: Test of the Effect of Hexafluoroisopropanol (HFIP) on the Detection Capacity of a Method Using a Pair of Antibodies Capable of Binding Specifically to TDP-43 NAFβ

The HFIP was used pure (100%) or diluted in lysis buffer to obtain 20%, 10% and 2% solutions.

The following reagents were distributed in a 384-well plate in the following order:
1) 8 μL of a sample of TDP-43 NAFβ prepared according to the protocol described in Example 2.
2) 8 μL of the different solutions of HFIP at 100%, 20%, 10% and 2% or of supplemented lysis buffer.
3) Addition of FRET detection reagents:
   2 μL of Donor antibody Ac1 prepared as described in Example 2
   2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 10 gives the signals obtained with the different tested concentrations of HFIP. For concentrations greater than 2%, HFIP decreases the detection signal of TDP-43 NAFβ by more than 75%. Its possible effect on aggregates will therefore be evaluated in this format with a concentration of 2%.

### Example 6: Test of HFIP as Disaggregating Agent of TDP-43 AFβ

The HFIP was diluted in lysis buffer to obtain a 2% solution.

The following reagents were distributed in a 384-well plate in the following order:
1) 8 μL of a sample of TDP-43 AFβ prepared according to the protocol described in example 2.
2) 8 μL of a 2% HFIP solution or lysis buffer.
3) Addition of FRET detection reagents:
   2 μL of Donor antibody Ac1 prepared as described in Example 2
   2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The Ratio of the signals obtained was calculated as follows from the FRET signals obtained on the samples:

Ratio of the signals=(sample signal treated with 2% HFIP)/(sample signal treated with lysis buffer).

FIG. 11 gives the results obtained. The signal ratio is less than or equal to 1 (0.89), which indicates that the 2% HFIP treatment does not increase the detection of PNAFβ in the sample containing TDP-43 AFβ. It can be concluded that the treatment with 2% HFIP does not allow to disaggregate, even partially, TDP-43 AFβ.

### Example 7: Test of the Effect of Urea on the Detection Capacity of a Method Using a Pair of Antibodies Capable of Binding Specifically to TDP-43 NAFβ

The urea was diluted in lysis buffer to obtain 7M, 3.5M, 1.75M and 0.88M solutions.

The following reagents were distributed in a 384-well plate in the following order:
1) 8 μL of a sample of TDP-43 NAFβ prepared according to the protocol described in Example 2.
2) 8 μL of different 7M, 3.5M, 1.75M and 0.88M Urea solutions or lysis buffer.
3) Addition of FRET detection reagents:
   2 μL of Donor antibody Ac1 prepared as described in Example 2
   2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 12 gives the signals obtained with the different tested concentrations of urea. For concentrations above 3.5M, Urea reduces the detection signal of TDP-43 NAFβ by more than 75%. Its possible effect on the aggregates will therefore be evaluated in this format with concentrations less than or equal to 3.5M.

### Example 8: Test of Urea as Disintegrating Agent of TDP-43 AFβ

The urea was diluted in supplemented lysis buffer to obtain 3.5 M, 1.75 M and 0.88 M solutions.

The following reagents were distributed in a 384-well plate in the following order:
1) 8 μL of a sample of TDP-43 AFβ prepared according to the protocol described in Example 2.
2) 8 μL of an urea solution at different concentrations (3.5 M, 1.75 M and 0.88 M) or of lysis buffer.
3) Addition of FRET detection reagents:
   2 μL of Donor antibody Ac1 prepared as described in Example 2.
   2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The Ratio of the signals obtained was calculated as follows from the FRET signals obtained on the samples:

Signal ratio=(sample signal treated with 3.5M urea)/(sample signal treated with supplemented lysis buffer).

FIG. 13 gives the results obtained. The signal ratio is less than or equal to 1 for all the Urea tested concentrations, which indicates that these treatments do not increase the detection of TDP-43 PNAFβ in the aggregate sample. It can be concluded that the treatment with urea concentrations less than or equal to 3.5M does not disaggregate, even partially, TDP-43 AFβ.

### Example 9: Test of the Effect of Guanidinium Chloride on the Detection Capacity of a Method Using a Pair of Antibodies Capable of Binding Specifically to TDP-43 NAFβ

Guanidinium chloride was diluted in supplemented lysis buffer to obtain 6 M, 3 M and 1.5 M solutions.

The following reagents were distributed in a 384-well plate in the following order:
1) 8 μL of a sample of TDP-43 NAFβ prepared according to the protocol described in Example 2.
2) 8 μL of the various Guanidinium Chloride solutions at 6 M, 3 M and 1.5 M or supplemented lysis buffer.
3) Addition of FRET detection reagents:
   2 μL of Donor antibody Ac1 prepared as described in Example 2
   2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 14 gives the signals obtained with the different tested concentrations of guanidinium chloride. Regardless of its concentration, Guanidinium Chloride reduces the detection signal of TDP-43 NAFβ by more than 75%. Its possible effect on the aggregates cannot therefore be evaluated in this format because this compound prevents the detection of TDP-43 NAFβ.

### Example 10: Test of the Effect of Formic Acid (FA) and Trifluoroacetic Acid (TFA) on the Detection Capacity of a Method Using a Pair of Antibodies Capable of Binding Specifically to TDP-43 NAFβ

FA and TFA were diluted in supplemented lysis buffer to obtain 20%, 10% and 2% solutions.

The following reagents were distributed in a 384-well plate in the following order:
1) 8 μL of a TDP-43 NAFβ prepared according to the protocol described in Example 2.
2) 8 μL of different solutions of FA or TFA at 20%, 10% and 2% or supplemented lysis buffer.
3) Addition of FRET detection reagents:
   2 μL of Donor antibody Ac1 prepared as described in Example 2
   2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 15 gives the signals obtained with the different tested concentrations of FA or

TFA. Regardless of their concentrations, these reagents reduce the detection signal of TDP-43 NAFβ by more than 75%. Their possible effect on the aggregates cannot therefore be evaluated in this format because they prevent the detection of TDP-43 NAFβ.

### Example 11: Test of the Effect of Formic Acid (FA) Followed by Neutralization with NaOH on the Detection Capacity of a Method Using a Pair of Antibodies Capable of Binding Specifically to TDP-43 NAFβ

The FA was diluted in supplemented lysis buffer to obtain 20% solutions.

The NaOH was diluted in a 450 mM HEPES buffer to obtain a 5N solution

The following reagents were distributed in a 96-well plate in the following order:
1) 60 μL of a sample of TDP-43 NAFβ prepared according to the protocol described in Example 2.
2) 8 μL of 20% FA solution or supplemented lysis buffer. The mixture was incubated for 15 minutes at room temperature. The pH measured at this step is equal to 2.5.
3) 9 μL of 5N NaOH solution or supplemented lysis buffer. The pH measured after these additions is equal to 7.6.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 16 compares the PNAFβ detection signal obtained with the FA then NaOH treatment with that obtained in the absence of treatment. The treatment causes an almost total disappearance of the TDP-43 NAFβ detection signal. Their possible effect on the disaggregation of TDP-43 AFβ cannot therefore be evaluated.

### Example 12: Test of the Effect of Trifluoroacetic Acid (TFA) Followed by Neutralization with NaOH on the Detection Capacity of a Method Using a Pair of Antibodies Capable of Binding Specifically to TDP-43 NAFβ

The TFA was diluted in supplemented lysis buffer to obtain 20% solutions.

The NaOH was diluted in a 450 mM HEPES buffer to obtain a 5N solution.

The following reagents were distributed in a 96-well plate in the following order:
1) 60 μL of a sample of TDP-43 NAFβ prepared according to the protocol described in Example 2.
2) 8 μL of a 20% TFA solution or supplemented lysis buffer. The mixture was incubated for 15 minutes at room temperature. The pH measured at this step is equal to 0.6.
3) 4.5 μL of 5N NaOH solution or supplemented lysis buffer. The pH measured after this addition is equal to 7.5.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 17 compares the PNAFβ detection signal obtained with the TFA then NaOH treatment with that obtained in the absence of treatment. Even if this treatment significantly affects the detection signal of TDP-43 NAFβ (˜62%), the remaining signal allows to test it on the disaggregation of TDP-43 AFβ.

### Example 13: Test of the Effect of Trifluoroacetic Acid (TFA) Followed by Neutralization with NaOH as Disintegrating Agent of TDP-43 AFβ

The TFA was diluted in supplemented lysis buffer to obtain 20% solutions.

The NaOH was diluted in a 450 mM HEPES buffer to obtain a 5 N solution.

The following reagents were distributed in a 96-well plate in the following order:
1) 60 μl of a sample of TDP-43 AFβ prepared according to the protocol described in Example 2.
2) 8 μL of a 20% TFA solution or a TFA/NaOH mixture (obtained by mixing 8 volumes of a 20% TFA solution with 4.5 volumes of a 5N NaOH solution). The mixture was incubated for 15 minutes at room temperature.
3) 4.5 μL of a 5N NaOH solution or a TFA/NaOH mixture. The pH measured after this addition is equal to 7.5.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The ratio of the signals obtained was calculated as follows from the FRET signals obtained on the samples:

Signal ratio=(Sample signal treated with 20% TFA then 5N NaOH)/(Sample signal treated with the TFA/NaOH mixture).

FIG. 18 gives the results obtained. The signal ratio is less than or equal to 1 (0.56), indicating that the treatment does not increase the detection of TDP-43 NAFβ in the aggregate sample. It can be concluded that the treatment with 20% TFA followed by neutralization with NaOH does not disaggregate, even partially, TDP-43 AFβ.

### Example 14: Test of the Effect of Formic Acid (FA) Followed by Neutralization with NH₄OH on the Detection Capacity of a Method Using a Pair of Antibodies Capable of Binding Specifically to TDP-43 NAFβ

The FA was diluted in supplemented lysis buffer to obtain 20% solutions.

The NH₄OH was diluted in a 450 mM HEPES buffer to obtain a 5N solution.

The following reagents were distributed in a 96-well plate in the following order:
1) 60 μL of a sample of TDP-43 NAFβ prepared according to the protocol described in Example 2.
2) 8 μL of 20% FA solution or supplemented lysis buffer. The mixture was incubated for 15 minutes at room temperature. The pH measured at this step is equal to 2.5.
3) 12 μL of a 5N NH₄OH solution. The pH measured after this addition is equal to 7.2.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 19 compares the TDP-43 NAFβ detection signal obtained with the FA then NH₄OH treatment with that obtained in the absence of treatment. Even if this treatment significantly affects the detection signal of TDP-43 NAFβ (˜68%), the remaining signal allows to test it on the disaggregation of TDP-43 AFβ.

### Example 15: Test of the Effect of Formic Acid (FA) Followed by Neutralization with NH₄OH as Disintegrating Agent of TDP-43 AFβ

The FA was diluted in supplemented lysis buffer to obtain 20% solutions.

The NH₄OH was diluted in a 450 mM HEPES buffer to obtain a 5N solution

The following reagents were distributed in a 96-well plate in the following order:
1) 60 μL of a sample of TDP-43 AFβ prepared according to the protocol described in Example 2
2) 8 μL of a 20% FA solution or an FA/NH₄OH mixture (obtained by mixing 8 volumes of a 20% TFA solution with 12 volumes of a 5N NH₄OH solution). The mixture was incubated for 15 minutes at room temperature.
3) 12 μL of a 5N NH₄OH solution or an FA/NH₄OH mixture. The pH measured after this addition is equal to 7.2.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The ratio of the signals obtained was calculated as follows from the FRET signals obtained on the samples:

Signal ratio=(Sample signal treated with 20% FA then 5N NH₄OH)/(Sample signal treated with the FA/NH₄OH mixture).

FIG. 20 gives the results obtained. The signal ratio is less than or equal to 1 (0.78) indicating that the treatment does not increase the detection of TDP-43 NAFβ in the aggregate sample. It can be concluded that the treatment with 20% FA followed by neutralization with NH₄OH does not disaggregate, even partially, TDP-43 AFβ.

### Example 16: Test of the Effect of Trifluoroacetic Acid (TFA) Followed by Neutralization with NH₄OH on the Detection Capacity of a Method Using a Pair of Antibodies Capable of Binding Specifically to TDP-43 NAFβ

The TFA was diluted in supplemented lysis buffer to obtain 20% solutions.

The NH₄OH was diluted in a 450 mM HEPES buffer to obtain a 5N solution.

The following reagents were distributed in a 96-well plate in the following order:
1) 60 μL of a sample of TDP-43 NAFβ prepared according to the protocol described in Example 2.
2) 8 μL of a 20% TFA solution or supplemented lysis buffer. The mixture was incubated for 15 minutes at room temperature. The pH measured at this step is equal to 0.6.
3) 7 μL of a 5N NH₄OH solution. The pH measured after this addition is equal to 7.5.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 21 compares the PNAFβ detection signal obtained with the TFA then NH₄OH treatment with that obtained in the absence of treatment. Even if this treatment significantly affects the detection signal of TDP-43 NAFβ (˜72%), the remaining signal allows to test it on the disaggregation of TDP-43 AFβ.

### Example 17: Test of the Effect of Trifluoroacetic Acid (TFA) Followed by Neutralization with NH₄OH as Disintegrating Agent of TDP-43 AFβ

The TFA was diluted in supplemented lysis buffer to obtain 20% solutions.

The NH₄OH was diluted in a 450 mM HEPES buffer to obtain a 5N solution.

The following reagents were distributed in a 96-well plate in the following order:
1) 60 μL of a sample of TDP-43 AFβ prepared according to the protocol described in example 2
2) 8 μL of a 20% TFA solution or a TFA/NH₄OH mixture (obtained by mixing 8 volumes of a 20% TFA solution with 7 volumes of a 5N NH₄OH solution). The mixture was incubated for 15 minutes at room temperature.
3) 7 μl of a 5N NH₄OH solution or a TFA/NH₄OH mixture. The pH measured after this addition is equal to 7.5.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The ratio of the signals obtained was calculated as follows from the FRET signals obtained on the samples:

Signal ratio=(Sample signal treated with 20% TFA then 5N NH₄OH)/(Sample signal treated with the TFA/NH₄OH mixture).

FIG. 22 gives the results obtained. The signal ratio is less than or equal to 1 (0.42) indicating that the treatment does not increase the detection of TDP-43 NAFβ in the aggregate sample. It can be concluded that the treatment with 20% TFA followed by neutralization with NH₄OH does not disaggregate, even partially, TDP-43 AFβ.

### Example 18: Effect of NaOH Solutions Giving pH Values Comprised Between 8.2 and 13.3 Followed by Neutralization with HCl on the Method Using TDP-43 NAFβ Detection Reagents

NaOH Tested Solutions

TABLE 4

| | | pH measured after addition to the |
|---|---|---|
| Condition | NaOH solution | TDP-43 NAFβ sample |
| A | 0.5N | 8.2 |
| B | 0.6N | 8.5 |
| C | 0.8N | 9.6 |
| D | 1.2N | 12.8 |
| E | 2.4N | 13.2 |
| F | 5N | 13.3 |

HCl Tested Solutions

TABLE 5

| Condition | HCl solution |
|---|---|
| A | 0.34N |
| B | 0.34N |
| C | 0.5N |
| D | 1N |
| E | 2N |
| F | 5N |

NaOH/HCl Tested Mixtures (Obtained by Mixing an Identical Volume of an NaOH Solution and an HCl Solution)

TABLE 6

| | | | pH measured after addition |
|---|---|---|---|
| Condition | NaOH solution | HCl solution | to the TDP-43 NAFβ sample |
| A | 0.5N | 0.34N | 7.6 |
| B | 0.6N | 0.34N | 7.4 |
| C | 0.8N | 0.5N | 7.4 |
| D | 1.2N | 1N | 7.2 |
| E | 2.4N | 2N | 7.4 |
| F | 5N | 5N | 7 |

The following reagents were distributed in a 96-well plate:
60 μL of a sample of TDP-43 NAFβ prepared according to the protocol described in Example 2.
10 μL of different NaOH solutions (A, B, C, D, E or F) or buffer. The mixture was incubated for 15 minutes at room temperature.
10 μL of different HCl solutions (A, B, C, D, E or F) or buffer.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 23 shows the results obtained with the different treatment modes. Treatment with an NaOH solution resulting in a pH of 13.3 followed by neutralization with HCl does not allow detection of TDP-43 NAFβ. The disintegrating power of all the other solutions could be tested on TDP-43 AFβ.

### Example 19: Determination of the Minimum and Maximum pH to Disaggregate TDP-43 AFβ with NaOH

NaOH Tested Solutions

TABLE 7

| | | pH measured after addition to the |
|---|---|---|
| Condition | NaOH solution | TDP-43 AFβ sample |
| A | 0.5N | 8.2 |
| B | 0.6N | 8.5 |
| C | 0.8N | 9.6 |
| D | 1.2N | 12.8 |
| E | 2.4N | 13.2 |

HCl Tested Solutions

TABLE 8

| Condition | HCl solution |
|---|---|
| A | 0.34N |
| B | 0.34N |
| C | 0.5N |
| D | 1N |
| E | 2N |

NaOH/HCl Tested Mixtures (Obtained by Mixing an Identical Volume of an NaOH Solution and an HCl Solution)

TABLE 9

| | | | pH measured after addition |
|---|---|---|---|
| Condition | NaOH solution | HCl solution | to the TDP-43 NAFβ sample |
| A | 0.5N | 0.34N | 7.6 |
| B | 0.6N | 0.34N | 7.4 |
| C | 0.8N | 0.5N | 7.4 |
| D | 1.2N | 1N | 7.2 |
| E | 2.4N | 2N | 7.4 |

The following reagents were distributed in a 96-well plate:
60 μL of a sample of TDP-43 AFβ prepared according to the protocol described in Example 2.
10 μL of different NaOH solutions (A, B, C, D or E) or corresponding NaOH/HCl mixtures (A, B, C, D or E). The mixture was incubated for 15 minutes at room temperature.
10 μL of different HCl solutions (A, B, C, D, E) or corresponding NaOH/HCl mixtures (A, B, C, D, E).

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The ratio of the signals obtained was calculated as follows from the FRET signals obtained on the samples:

Ratio of the signals=(Sample signal treated with NaOH then HCl)/(Sample signal treated with NaOH/HCl mixture).

FIG. 24 shows the results obtained as a function of the pH induced by the various NaOH tested solutions. The detection of TDP-43 NAFβ is possible when the pH ranges from 8.5 to 13.2 during the disaggregation step. However, the pH allowing an optimal amplitude for detecting TDP-43 NAFβ is around pH 12.8.

### Example 20: Determination of the Time Required to Disaggregate TDP-43 AFβ at a pH of 12.8

The following reagents were distributed in a 96-well plate in the following order:
1) 60 μL of a sample of TDP-43 AFβ prepared according to the protocol described in Example 2
2) 10 μL of a 1.2N NaOH solution (pH of the sample brought to 12.8) or of an NaOH/HCl mixture (obtained by mixing an identical volume of a 1.2N NaOH solution and a 1N HCl solution). The mixture was incubated between 30 seconds and 1 hour at room temperature depending on the wells.
3) 10 μL of a 1N HCl solution (pH of the sample brought to 7.5) in the wells treated with 1.2N NaOH or 10 μL of the NaOH/HCl mixture in the wells treated with the NaOH/HCl mixture.

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The ratio of the signals obtained was calculated as follows from the FRET signals obtained on the samples:

Ratio of the signals=(Sample signal treated with 1.2N NaOH then 1N HCl)/(Sample signal treated with NaOH/HCl mixture).

FIG. 25 gives the results obtained as a function of the time of contact with 1.2N NaOH. The results show that the 1.2N NaOH treatment allowed to at least partially disaggregate TDP-43 AFβ independently of the contact time (ratio of the signals always greater than 1). However, the best disaggregation was obtained for contact times ranging from 5 minutes to 30 minutes.

### Example 21: Determination of the Minimum and Maximum pH when Measuring the Detection of TDP-43 NAFβ

NaOH Tested Solutions

1.2N NaOH solution (allows to bring the pH of the sample to 12.8)

HCl Tested Solutions

TABLE 10

| Condition | HCl solution |
|---|---|
| A | 1.4N |
| B | 1.3N |
| C | 1.2N |
| D | 1N |
| E | 0.7N |
| F | 0.6N |
| G | 0.5N |
| H | 0.4N |
| I | 0.3N |

NaOH/HCl Tested Mixtures (Obtained by Mixing an Identical Volume of an NaOH Solution and an HCl Solution).

TABLE 11

| | | | pH measured after addition |
|---|---|---|---|
| Condition | NaOH solution | HCl solution | to the TDP-43 AFβ sample |
| A | 1.2N | 1.4N | 5.7 |
| B | 1.2N | 1.3N | 6 |
| C | 1.2N | 1.2N | 6.4 |
| D | 1.2N | 1N | 7.2 |
| E | 1.2N | 0.7N | 8 |
| F | 1.2N | 0.6N | 8.4 |
| G | 1.2N | 0.5N | 10.4 |
| H | 1.2N | 0.4N | 11.2 |
| I | 1.2N | 0.3N | 12.8 |

The following reagents were distributed in a 96-well plate:
60 μL of a sample of TDP-43 AFβ prepared according to the protocol described in example 2.
10 μL of different 1.2N NaOH solutions or corresponding NaOH/HCl mixtures (A, B, C, D, E, F, G, H or I). The mixture was incubated for 15 minutes at room temperature.
10 μL of different HCl solutions (A, B, C, D, E, F, G, H or I) or corresponding NaOH/HCl mixtures (A, B, C, D, E, F, G, H or I).

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The Ratio of the signals obtained was calculated as follows from the FRET signals obtained on the samples:

Signal ratio=(Sample signal treated with 1.2N NaOH then HCl)/(Sample signal treated with NaOH/HCl mixture).

FIG. 26 shows that TDP-43 NAFβ can be detected after treatment at pH=12.8 when the pH is then brought back between pH=6 and pH=10.5. Detection is optimal when the pH is comprised between 6 and 8.5.

### Example 22: Measurement of the Level of Aggregation of a Beta Amyloid Peptide 1-42 Using NaOH as Disaggregating Agent and HCl as Neutralizing Agent

The method described below is based on HTRF technology (see Example 1 for principle).

The 1.2N NaOH and 1N HCl solutions were prepared as described in the previous examples.

Preparation of samples containing monomeric (1-42 NAFβ) or aggregated (1-42 AFβ) beta 1-42 amyloid peptide: freeze-dried human beta 1-42 amyloid peptide (ERI275BAS, The ERI Amyloid Laboratory, LLC, Oxford) was re-suspended according to the supplier's recommendations then diluted in 10 mM Sodium Phosphate pH 7.4 buffer to a concentration of 30 μM (1-42 NAFβ). An identical solution of beta 1-42 amyloid peptide is incubated for 188 hours at 25° C. to obtain 1-42 AFβ. The 2 samples were frozen at −80° C. before future use. Before use, the level of aggregation of the 2 samples was checked by the Thioflavin T method.

On the day of the test, the samples 1-42 NAFβ and 1-42 AFβ were thawed then diluted in 10 mM sodium phosphate buffer pH 7.4 at a concentration of 2.4 ng/mL before distribution in the microplate.

The following reagents were distributed in a 384-well plate in the following order:
1) 12 μL of a 1-42 AFβ sample or a 1-42 NAFβ sample.
2) 2 μL of a 1.2N NaOH solution or an NaOH/HCl mixture (obtained by mixing an equal volume of a 1.2N NaOH solution and a 1N HCl solution). The mixture was incubated for 15 minutes at room temperature.
3) 2 μL of a 1N HCl solution or an NaOH/HCl mixture.
4) Addition of FRET detection reagents:
   2 μL of donor antibody prepared as described in Example 4.
   2 μL of Acceptor antibody prepared as described in Example 4.

The plates were incubated overnight at a temperature comprised between 2° C. and 8° C.

The detection of the HTRF signal in the various plates was carried out on a PHERAstar FS Lamp apparatus (BMG Labtech) using an HTRF detection module.

The Aggregation Ratio is calculated as follows from the HTRF signals obtained on the samples:

Signal ratio=(Sample signal treated with 1.2N NaOH then 1N HCl)/(Sample signal treated with the NaOH/HCl mixture).

FIG. 27 shows the signal ratios obtained with samples 1-42 NAFβ and 1-42 MB. The signal ratio obtained on the 1-42 AFβ sample is much higher than that obtained on the 1-42 NAFβ sample. This result indicates that the method is able to measure the level of 1-42 amyloid peptide aggregation.

### Example 23: Effect of a Treatment with NaOH, Potassium Hydroxide (KOH) or NH₄OH Followed by Neutralization with HCl on the Detection Capacity of a Method Using a Pair of Antibodies Able to Bind Specifically to TDP-43 NAFβ

Preparation of NaOH, KOH, NH₄OH and HCl Solutions to Treat TDP43-AFβ Lysates

TABLE 12

| | | pH measured after addition to the |
|---|---|---|
| Condition | Base solution | TDP-43 NAFβ sample |
| A | NaOH 1.2N | 12.8 |
| B | KOH 1.2N | 12.5 |
| C | NH₄OH 10N | 10.9 |
| D | NaOH 1.2N | 9.6 |
| E | NH₄OH 1.2N | 9.6 |

HCl Tested Solutions

TABLE 13

| | | pH measured after addition to the |
|---|---|---|
| Condition | HCl solution | TDP-43 NAFβ sample |
| A | 1N | 7.2 |
| B | 0.67N | 7.5 |
| C | 6.6N | 7.4 |
| D | 0.5N | 7.4 |
| E | 0.5N | 7.6 |

Base/HCl Tested Mixtures (Obtained by Mixing an Identical Volume of an NaOH Solution and an HCl Solution).

TABLE 14

| | | | pH measured after addition |
|---|---|---|---|
| Condition | Base solution | HCl solution | to the TDP-43 NAFβ sample |
| A | NaOH 1.2N | 1N | 7.2 |
| B | KOH 1.2N | 0.67N | 7.5 |
| C | NH₄OH 10N | 6.6N | 7.4 |
| D | NaOH 1.2N | 0.5N | 7.4 |
| E | NH4OH 1.2N | 0.5N | 7.6 |

The following reagents were distributed in a 96-well plate:
60 μL of a sample of TDP-43 NAFβ prepared according to the protocol described in Example 2.
10 μL of different Base solutions (A, B, C, D or E) or corresponding Base/HCl mixtures (A, B, C, D or E). The mixture was incubated for 15 minutes at room temperature.
10 μL of different HCl solutions (A, B, C, D, E) or corresponding Base/HCl mixtures (A, B, C, D, E).

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

FIG. 28 shows the TDP-NAFβ detection signals obtained with the different treatments. The detection of TDP-43 NAFβ is possible regardless of the bases used when a neutralization step with HCl allowing a pH comprised between 7 and 8 is carried out. The disintegrating effect of these bases will therefore be tested on TDP-43 AFβ.

### Example 24: Effect of a Treatment with NaOH, Potassium Hydroxide (KOH) or NH₄OH Followed by Neutralization with HCl on the Disaggregation of TDP-43 AFβ

Preparation of NaOH, KOH, NH₄OH and HCl Solutions to Treat TDP43-AFβ Lysates

TABLE 15

| | | pH measured after adding TDP-43 |
|---|---|---|
| Condition | Base solution | AFβ to the sample |
| A | NaOH 1.2N | 12.8 |
| B | KOH 1.2N | 12.5 |
| C | NH₄OH 10N | 10.9 |
| D | NaOH 1.2N | 9.6 |
| E | NH₄OH 1.2N | 9.6 |

HCl Tested Solutions

TABLE 16

| | | pH measured after addition to the |
|---|---|---|
| Condition | HCl solution | TDP-43 AFβ sample |
| A | 1N | 7.2 |
| B | 0.67N | 7.5 |
| C | 6.6N | 7.4 |
| D | 0.5N | 7.4 |
| E | 0.5N | 7.6 |

Base/HCl Tested Mixtures (Obtained by Mixing an Identical Volume of an NaOH Solution and an HCl Solution)

TABLE 17

| | | | pH measured after addition |
|---|---|---|---|
| Condition | base solution | HCl solution | to the TDP-43 AFβ sample |
| A | NaOH 1.2N | 1N | 7.2 |
| B | KOH 1.2N | 0.67N | 7.5 |
| C | NH₄OH 10N | 6.6N | 7.4 |
| D | NaOH 1.2N | 0.5N | 7.4 |
| E | NH₄OH 1.2N | 0.5N | 7.6 |

The following reagents were distributed in a 96-well plate:
60 μL of a sample of TDP-43 AFβ prepared according to the protocol described in example 2.
10 μL of different Base solutions (A, B, C, D or E) or corresponding Base/HCl mixtures (A, B, C, D or E). The mixture was incubated for 15 minutes at room temperature.
10 μL of different HCl solutions (A, B, C, D, E) or corresponding Base/HCl mixtures (A, B, C, D, E).

A portion of the volume contained in the wells of the 96-well plate was transferred to a 384-well plate before adding the FRET detection reagents with:
16 μL of the mixture from the 96-well plate
2 μL of Donor antibody Ac1 prepared as described in Example 2
2 μL of Acceptor antibody Ac2 prepared as described in Example 2.

The plates were incubated overnight at room temperature. The detection of the HTRF signal in the different plates was carried out on a PHERAstar FS Lamp apparatus (BMG Labtech) using an HTRF detection module.

The Ratio of the signals obtained was calculated as follows from the FRET signals obtained on the samples:

Ratio of the signals=(Sample signal treated with Base then HCl)/(Sample signal treated with Base/HCl mixture).

FIG. 29 shows the ratio of the signals obtained with the various treatments. The two NaOH treatment conditions (1.2N and 0.8N), respectively giving pH values of 12.8 and 9.6, allow to detect TDP43-NAFβ, confirming their disaggregating effect on TDP-43 AFβ. Treatment with 1.2N KOH (pH=12.5) allows weak detection of TDP43-NAFβ showing a weak disaggregating effect of TDP-43 AFβ. The 2 treatments with NH₄OH (10N and 1.2N) which give pH values similar to the NaOH treatments do not allow to detect TDP43-NAFβ. This result shows that NH₄OH has no disaggregating effect of TDP-43 AFβ.

### Example 25: Method Allowing to Identify Pairs of Antibodies Capable of Binding Specifically to Alpha-Synuclein NAFβ (Alpha-Syn NAFβ)

The method is based on FRET technology (HTRF® technology from Cisbio Bioassays), as detailed in Example 1.

Pair of Tested Antibodies

A pair of antibodies directed against Alpha-Synuclein labeled respectively with the Donor and with the Acceptor is that contained in the HTRF Total-Alpha-Synuclein kit marketed by Cisbio Bioassays (ref. 6FNSYPEG). Each antibody was diluted in the kit diluent as recommended by the user manual.

Preparation of Alpha-Syn NAFβ and Alpha-Syn NAFβ Samples

Alpha-Syn NAFβ and Alpha-Syn AFβ were obtained from StressMarq (StressMarq Active Human recombinant A53T Mutant Alpha Synuclein Protein Monomer, ref SPR-325 and Active Human recombinant A53T Mutant Alpha Synuclein Protein Preformed Fibrils Type 1 ref. SPR-326). They were diluted to 15.6 ng/mL in the lysis buffer of the HTRF Total-A Synuclein kit.

Test of the Pair of Antibodies on Alpha-Syn NAFβ and Alpha-Syn

The following reagents were distributed in a 384-well plate in the following order:
1) 12 μL of Alpha-Syn NAβ or Alpha-Syn AFβ at 15.6 ng/mL
2) 2 μL of lysis buffer. The mixture was incubated for 15 minutes at room temperature.
3) 2 μL of lysis buffer.
4) 2 μL of Donor antibody.
5) 2 μL of Acceptor antibody.

The detection of the FRET signal in the different plates was carried out on a PHERAstar FS Lamp apparatus (BMG Labtech) using an HTRF detection module.

The signals obtained in FRET on the Alpha-Syn NAFβ sample (Monomers) and on the Alpha-Syn AFβ sample (Aggregates) were compared by calculating the ratio of the FRET signals (Monomers/Aggregates) as shown in FIG. 30.

The ratio of the FRET (Monomer/Aggregate) signals of the pair of antibodies is greater than 2 (value of 6.64). This indicates that this pair of antibodies is able to specifically bind Alpha-Syn NAFβ over Alpha-Syn AFβ.

### Example 26: Effect of a 1.2N NaOH Solution Followed by Neutralization with 1N HCl on the Detection Capacity of a Method Using a Pair of Antibodies Capable of Binding Specifically to Alpha-Syn NAFβ

The Alpha-Syn NAFβ (StressMarq, Active Human recombinant A53T Mutant Alpha Synuclein Protein Monomer, ref. SPR-326) was diluted in the lysis buffer of the HTRF Total-Alpha Synuclein kit (Cisbio Bioassays, ref. 6FNSYPEG) at 15.6 ng/mL. Donor and acceptor antibodies in the HTRF Total-A Synuclein Kit were diluted as recommended by the supplier in the kit manual.

The following reagents were distributed in a 384-well plate in the following order:
1) 12 μL of Alpha-Syn NAFβ at 15.6 ng/mL
2) 2 μL of a 1.2N NaOH solution (pH of the sample brought to 12.8) or of an NaOH/HCl mixture (obtained by mixing an identical volume of a 1.2N NaOH solution and a 1N HCl solution). The mixture was incubated for 15 minutes at room temperature.
3) 2 μL of a 1N HCl solution (pH of the sample brought to 7.5) in the wells treated with 1.2N NaOH or 2 μL of the NaOH/HCl mixture in the wells treated with the NaOH/HCl mixture.
4) 2 μL of Donor antibody prepared as described in Example 25
5) 2 μL of Acceptor antibody prepared as described in Example 25

The plates were incubated overnight at room temperature. The detection of the HTRF signal in the different plates was carried out on a PHERAstar FS Lamp apparatus (BMG Labtech) using an HTRF detection module.

FIG. 31 gives the results obtained. They show that treatment with 1.2N NaOH followed by neutralization with 1N HCl has little effect on the detection of Alpha-Syn NAFβ by antibodies. The effect of this treatment in separating out Alpha-Syn AFβ can therefore be assessed.

### Example 27: Measurement of the Level of Aggregation of Alpha-Synuclein Using NaOH as Disintegrating Agent and HCl as Neutralizing Agent

The method is based on FRET technology (HTRF® technology from Cisbio Bioassays), as detailed in Example 1.

Pair of Tested Antibodies

The antibodies directed against Alpha-Synuclein labeled respectively with the Donor and with the Acceptor are those contained in the HTRF Total-Alpha-Synuclein kit marketed by Cisbio Bioassays (ref. 6FNSYPEG). They were diluted in the kit diluent as recommended by the user manual.

Preparation of Alpha-Syn NAFβ and Alpha-Syn NAFβ Samples

The Alpha-Syn NAFβ and the Alpha-Syn AFβ were obtained from the company StressMarq (StressMarq Active Human recombinant A53T Mutant Alpha Synuclein Protein Monomer, ref SPR-325 and Active Human recombinant A53T Mutant Alpha Synuclein Protein Preformed Fibrils Type 1 ref SPR-326). They were diluted to 15.6 ng/mL in the lysis buffer of the HTRF Total-Alpha Synuclein kit.

Test of the Pair of Antibodies on Alpha-Syn NAFβ and Alpha-Syn

The following reagents were distributed in a 384-well plate in the following order:
1) 12 μL of Alpha-Syn NAFβ or Alpha-Syn AFβ at 15.6 ng/mL
2) 2 μL of a 1.2N NaOH solution (pH of the sample brought to 12.8) or of an NaOH/HCl mixture (obtained by mixing an identical volume of a 1.2N NaOH solution and a 1N HCl solution). The mixture was incubated for 15 minutes at room temperature.
3) 2 μL of a 1N HCl solution (pH of the sample brought to 7.5) in the wells treated with 1.2N NaOH or 2 μL of the NaOH/HCl mixture in the wells treated with the NaOH/HCl mixture.
4) 2 μL of Donor antibody.
5) 2 μL of Acceptor antibody.

The detection of the FRET signal in the various plates was carried out on a PHERAstar FS Lamp apparatus (BMG Labtech) using an HTRF detection module.

FIG. 32 shows the signal ratios obtained with the Alpha-Syn NAFβ and Alpha-Syn AFβ samples. The signal ratio obtained on the Alpha-Syn AFβ sample is much higher than that obtained on the Alpha-Syn NAFβ sample. This result tells us that the method according to the invention allows to measure the level of aggregation of Alpha-Synuclein.

### BRIEF DESCRIPTION OF THE DRAWINGS

FIG. 1 illustrates the general principle of the method according to the invention. The comparison of the PNAFβ levels (Ratio of the signals) therefore allows to know whether the tested sample comprises a PAFβ. The ratio of the signals for a sample not comprising PAFβ will be equal to 1. The ratio of the signals for a sample comprising PAFβ will be greater than 1.

FIG. 2 shows the ELISA signal (O.D.) obtained for different dilutions of PNAFβ. The O.D. value corresponding to 80% of the maximum signal is used to determine the reference dilution to be used in the rest of the experiment.

FIG. 3 shows the results obtained in an ELISA test aiming at determining whether or not a pair of antibodies selectively recognizes a PNAFβ. A) PNAFβ non-selective pair of antibodies. B) PNAFβ selective pair of antibodies.

FIG. 4 shows the principle of an HTRF immunoassay. This type of immunoassay allows to know whether two antibodies, respectively labeled with the Donor and the Acceptor, are capable of creating a pair of antibodies capable of simultaneously recognizing an antigen of interest. A) the 2 tested antibodies are not able to create a pair of antibodies capable of recognizing the antigen of interest; there is no energy transfer between the Donor and the Acceptor and therefore no fluorescence signal emitted by the Acceptor. B) The two tested antibodies are capable of simultaneously recognizing the antigen of interest. A transfer of energy then occurs between the Donor and the Acceptor. This results in a specific fluorescence emission at 665 nm which is emitted by the acceptor.

FIG. 5 shows the HTRF signal obtained for different dilutions of PNAFβ. The HTRF signal value corresponding to 80% of the maximum signal is used to determine the reference dilution to be used in the rest of the experiment.

FIG. 6 shows the results obtained in an HTRF test aiming at determining whether or not a pair of antibodies selectively recognizes a PNAFβ. A) PNAFβ non-selective pair of antibodies. B) PNAFβ selective pair of antibodies.

FIG. 7 shows the calculation of the signal ratio between a TDP-43 AFβ aggregate sample and a TDP-43 NAFβ monomer sample obtained with a pair of antibodies directed against the TDP-43 protein. A signal ratio greater than 2 indicates that the tested pair is selective for TDP-43 NAFβ.

FIG. 8 shows the calculation of the signal ratio between an aggregate sample of beta 1-40 amyloid peptide and a monomer sample of this same peptide obtained with a pair of antibodies directed against the beta 1-40 amyloid peptide. The value of the signal ratio greater than 2 indicates that the tested pair is selective for the 1-40 amyloid peptide NAFβ (monomeric form).

FIG. 9 shows the calculation of the signal ratio between an aggregate sample of beta 1-42 amyloid peptide and a monomeric sample of this same peptide obtained with a pair of antibodies directed against the beta 1-42 amyloid peptide. The value of the signal ratio greater than 2 indicates that the tested pair is selective for the 1-42 amyloid peptide NAFβ (monomeric form).

FIG. 10 shows the effect of Hexafluoroisopropanol (HFIP) on the detection capacity of an HTRF method using a pair of antibodies capable of binding specifically to TDP-43 NAFβ.

FIG. 11 shows the absence of disintegrating effect of Hexafluoroisopropanol (HFIP) on a sample of TDP-43 AFβ.

FIG. 12 shows the effect of Urea on the detection capacity of an HTRF method using a pair of antibodies capable of binding specifically to TDP-43 NAFβ.

FIG. 13 shows the absence of disintegrating effect of Urea on a sample of TDP-43 AFβ.

FIG. 14 shows the effect of Guanidinium Chloride on the detection capacity of an HTRF method using a pair of antibodies capable of binding specifically to TDP-43 NAFβ.

FIG. 15 shows the effect of Formic Acid (A) or TFA (B) on the detection capacity of an HTRF method using a pair of antibodies capable of binding specifically to TDP-43 NAFβ.

FIG. 16 shows the effect of Formic Acid followed by neutralization with NaOH on the detection capacity of an HTRF method using a pair of antibodies capable of binding specifically to TDP-43 NAFβ.

FIG. 17 shows the effect of TFA followed by neutralization with NaOH on the detection capacity of an HTRF method using a pair of antibodies capable of binding specifically to TDP-43 NAFβ.

FIG. 18 shows the absence of disintegrating effect of TFA followed by neutralization with NaOH on a sample of TDP-43 AFβ.

FIG. 19 shows the effect of Formic Acid followed by neutralization with NH₄OH on the detection capacity of an HTRF method using a pair of antibodies capable of binding specifically to TDP-43 NAFβ.

FIG. 20 shows the absence of disintegrating effect of Formic Acid followed by neutralization with NH₄OH on a sample of TDP-43 AFβ.

FIG. 21 shows the effect of TFA followed by neutralization with NH₄OH on the detection capacity of an HTRF method using a pair of antibodies capable of binding specifically to TDP-43 NAFβ.

FIG. 22 shows the absence of disintegrating effect of TFA followed by neutralization with NH₄OH on a sample of TDP-43 AFβ.

FIG. 23 shows the effect of NaOH solutions giving pH values comprised between 8.2 and 13.3 followed by neutralization with HCl on the detection capacity of an HTRF method using a pair of antibodies capable of binding specifically to the TDP-43 NAFβ.

FIG. 24 shows the determination of the minimum and maximum pH to disaggregate a sample of TDP-43 AFβ with NaOH solutions.

FIG. 25 shows the determination of the time required to disaggregate a sample of TDP-43 AFβ with a solution of NaOH at pH=12.8.

FIG. 26 shows the determination of the minimum and maximum pH when measuring the detection of TDP-43 NAFβ.

FIG. 27 shows the measurement of the level of aggregation of a beta 1-42 amyloid peptide using NaOH as a disaggregating agent followed by neutralization with HCl.

FIG. 28 shows the effect of NaOH, KOH and NH₄OH solutions followed by HCl neutralization on the detection ability of an HTRF method using a pair of antibodies capable of specifically binding to TDP-43 NAFβ.

FIG. 29 shows the effect of solutions of NaOH, KOH and NH₄OH followed by neutralization with HCl on the disaggregation of TDP-43 NAFβ.

FIG. 30 shows the calculation of the signal ratio between an aggregate sample of Alpha-Synuclein (Alpha-Syn AFβ) and a monomer sample of this same protein (Alpha-Syn NAFβ) obtained with a pair of antibodies directed against Alpha-Synuclein. The value of the signal ratio greater than 2 indicates that the tested pair is selective for Alpha-Syn NAFβ.

FIG. 31 shows the effect of an NaOH solution followed by neutralization with HCl on the detection capacity of an HTRF method using a pair of antibodies capable of binding specifically to Alpha-Syn NAFβ.

FIG. 32 shows the measurement of the level of aggregation of Alpha-Synuclein using NaOH as a disaggregating agent followed by neutralization with HCl.

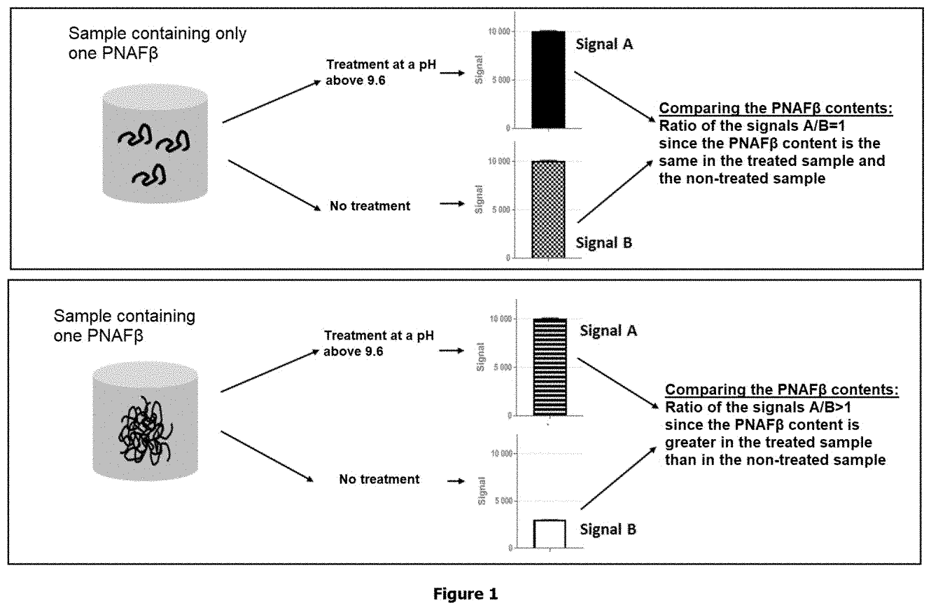

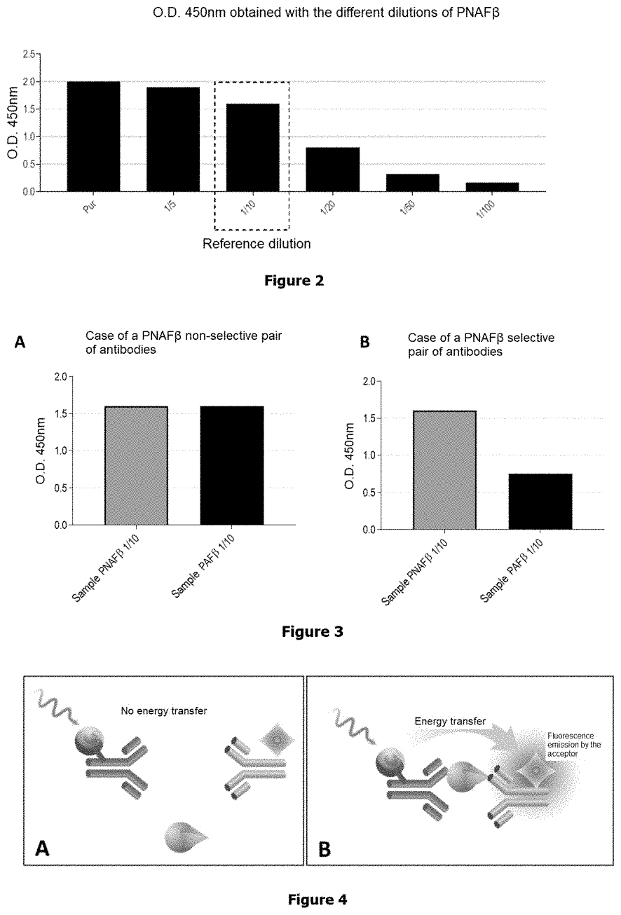

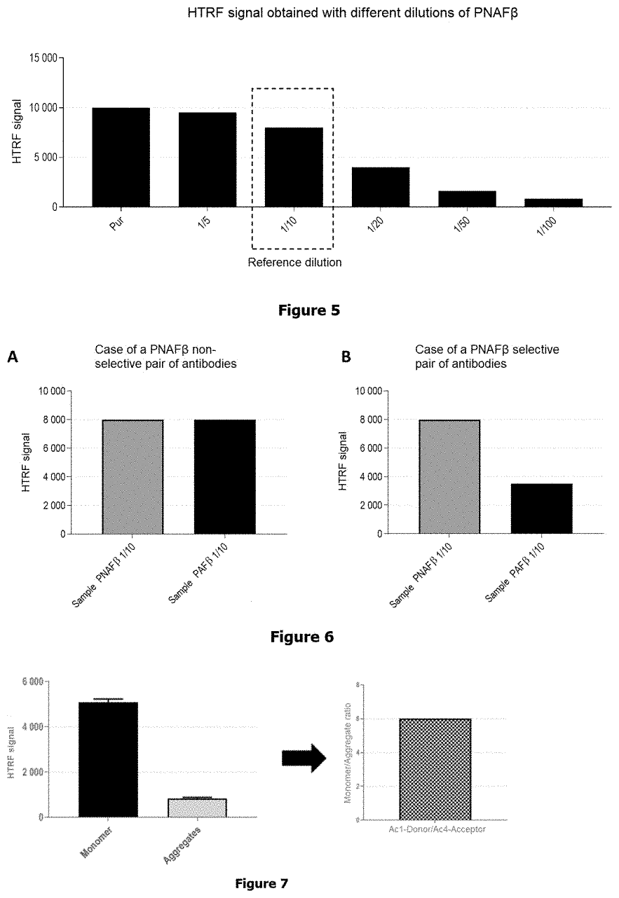

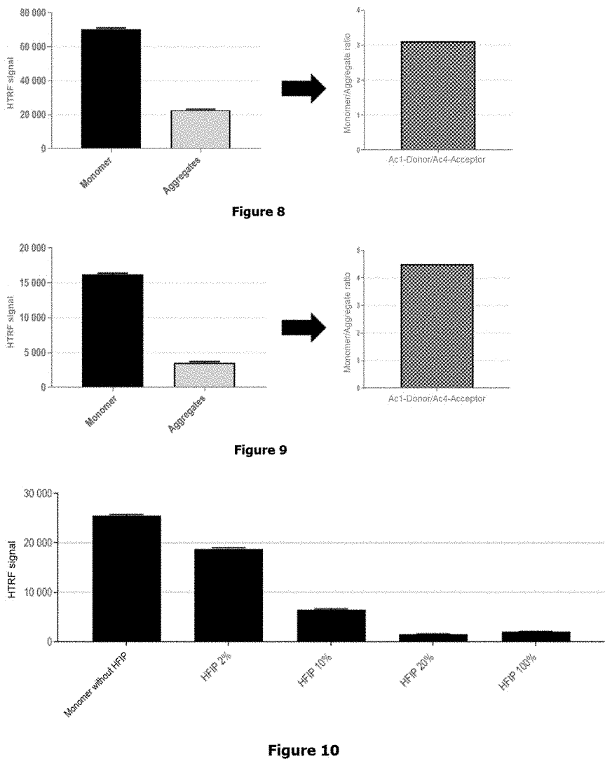

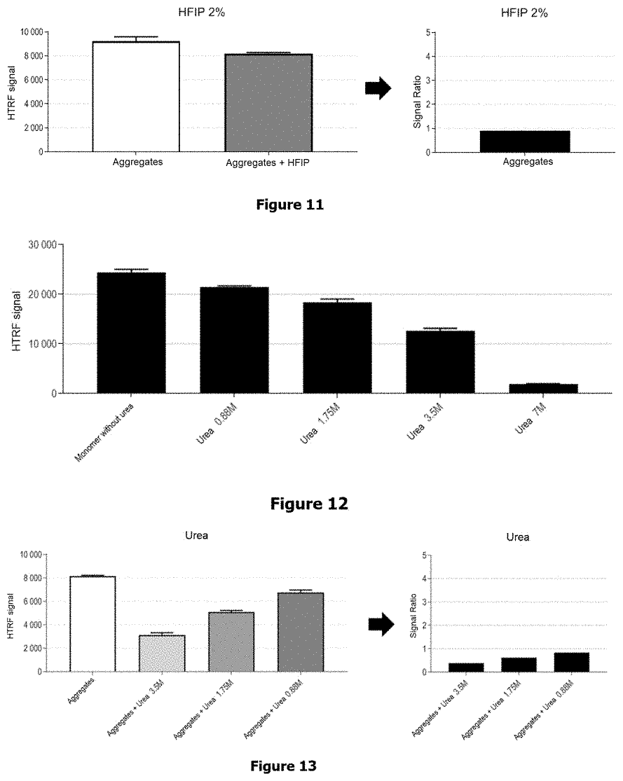

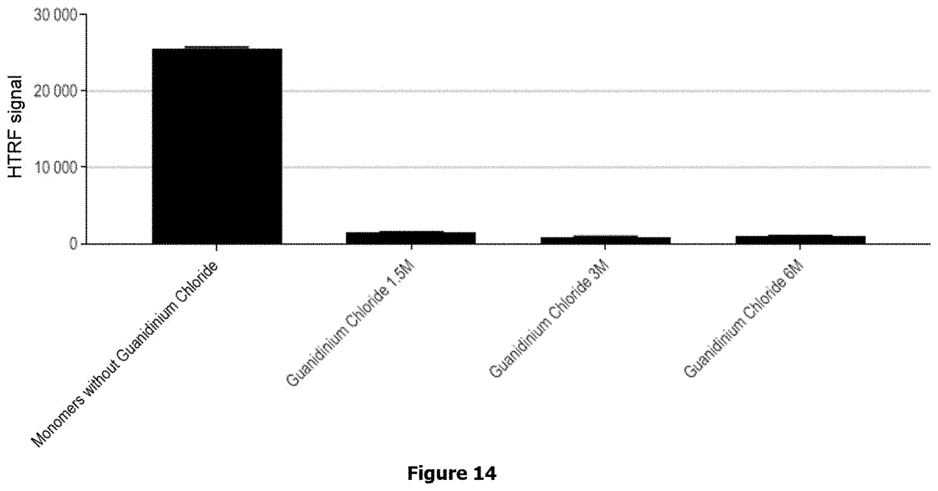

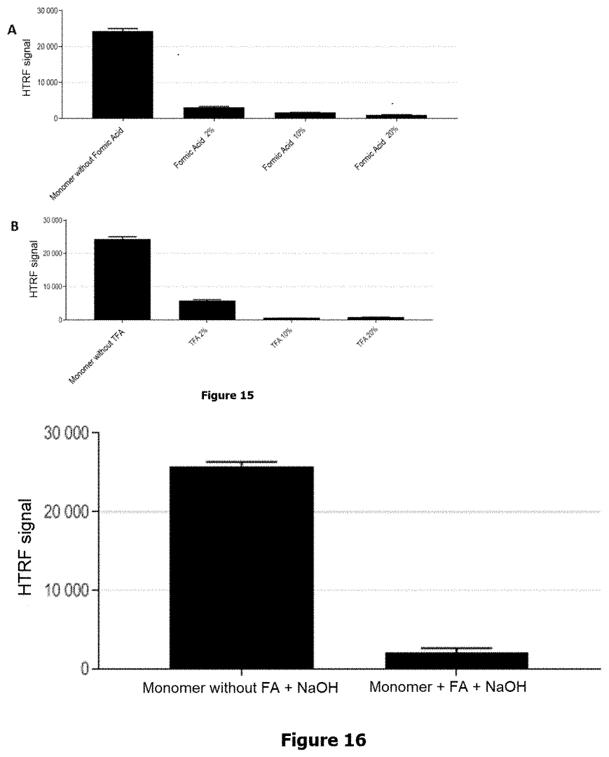

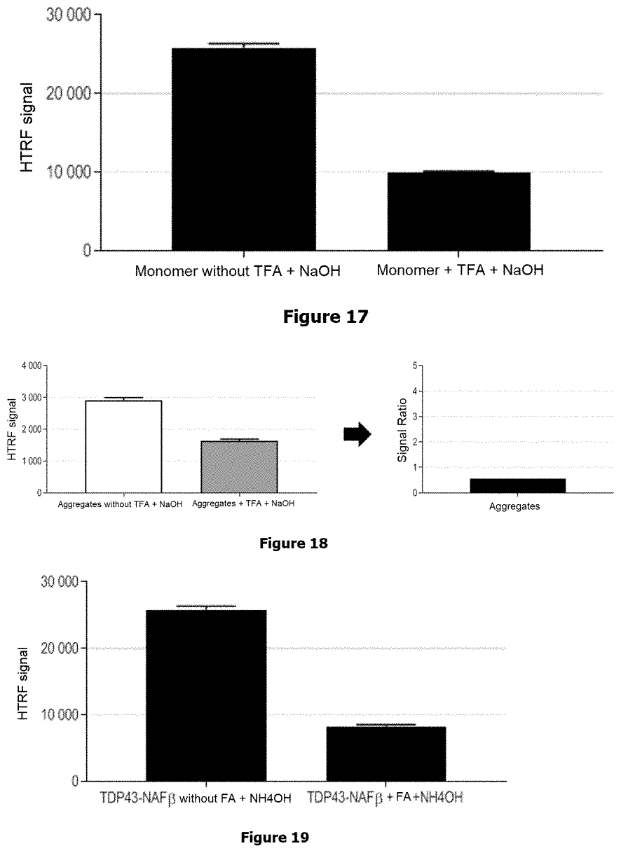

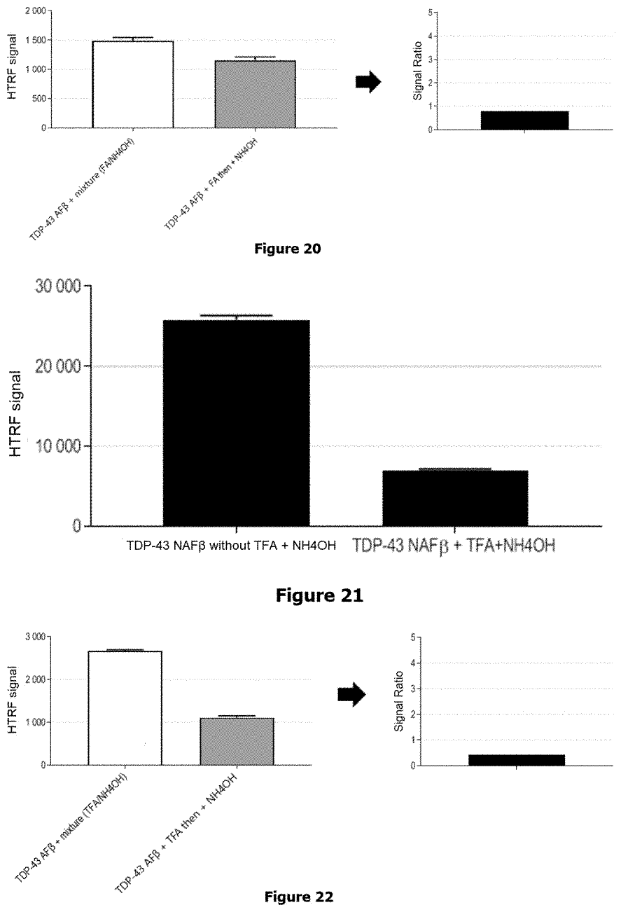

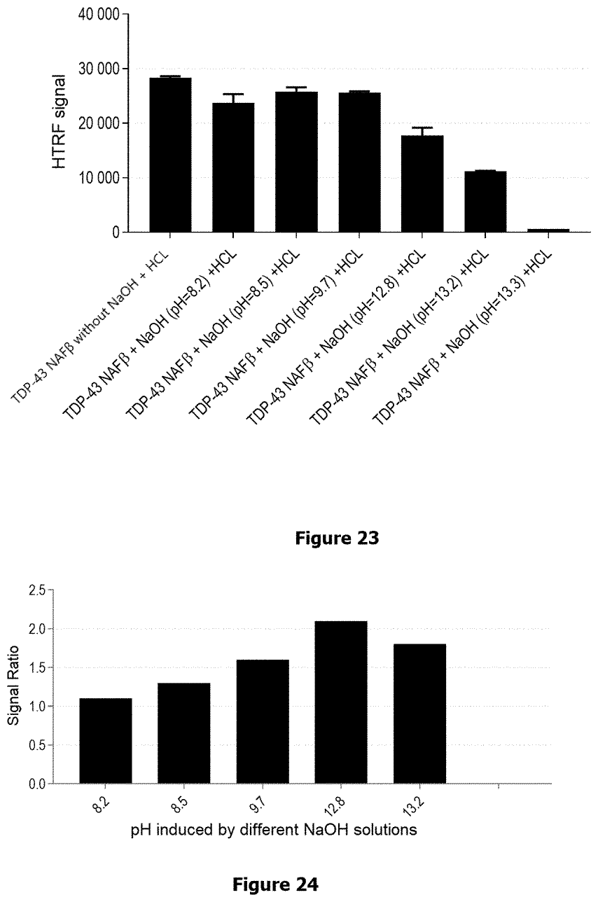

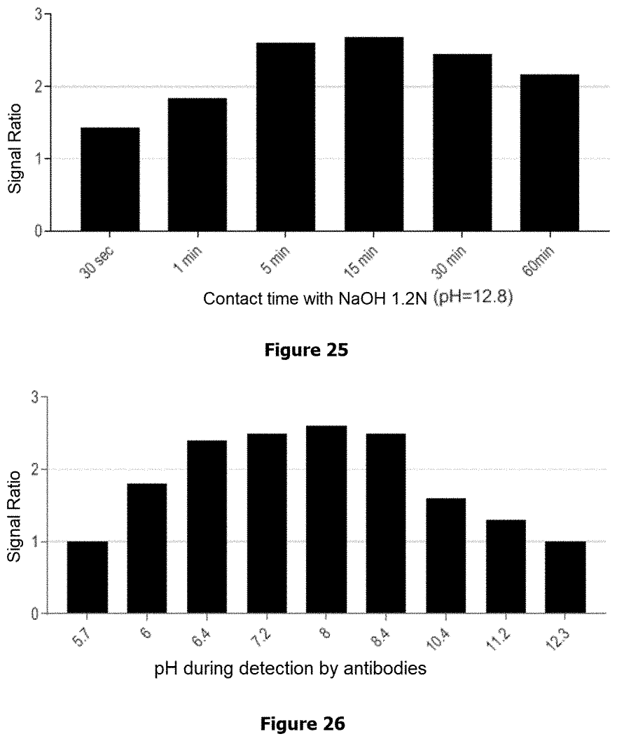

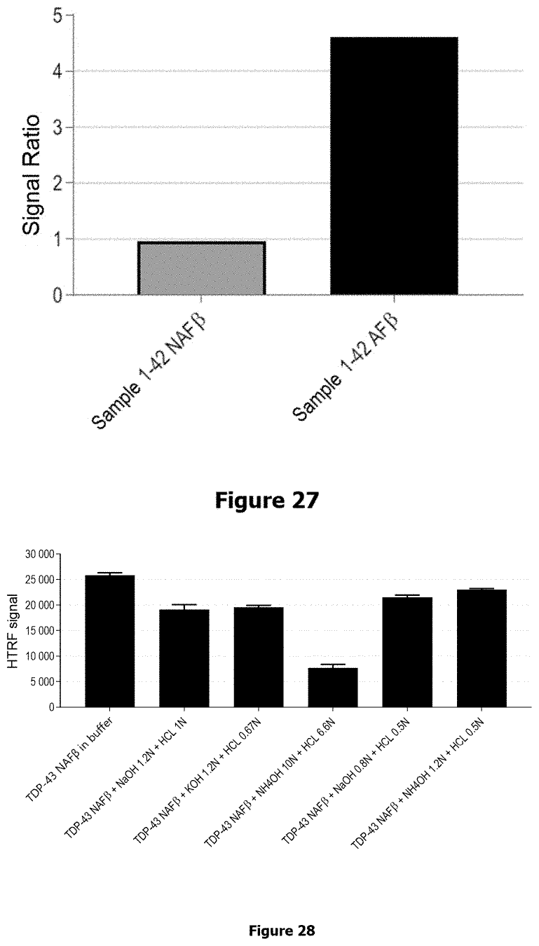

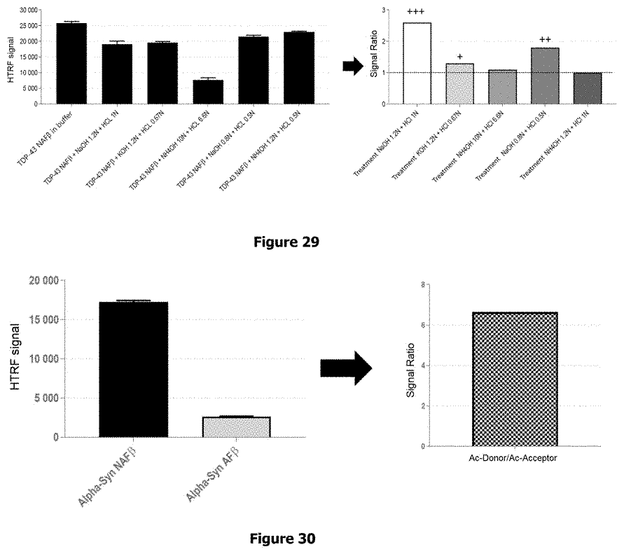

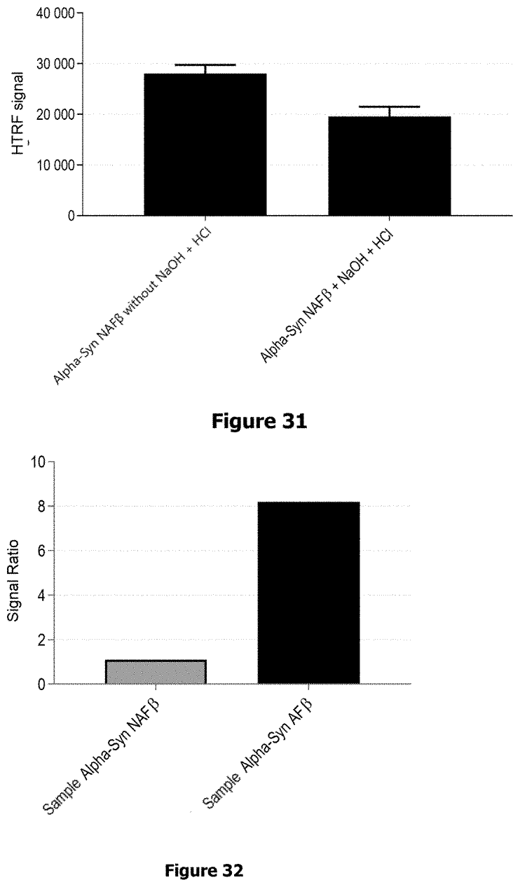

### BIBLIOGRAPHIC REFERENCES

[1] Harrison et al, RNA-binding proteins with prion-like domains in health and disease (2017) Biochem J.; 474(8): 1417-1438. doi:10.1042/BCJ20160499
[2] Chang et al., Detection and quantification of TAU aggregation using a membrane filter assay, Analytical Biochemistry 373 (2008) 330-336
[3] Howlett et al, Inhibition of fibril formation in b-amyloid peptide by a novel series of benzofuran, Biochem. J. (1999) 340, 283-289.
[4] Linghagen-Persson et al., Amyloid-b Oligomer Specificity Mediated by the IgM Isotype—Implications for a Specific Protective Mechanism Exerted by Endogenous Auto-Antibodies, PLoS ONE, November 2010|Volume 5|Issue 11|e13928
[5] Englund et al., Sensitive ELISA detection of amyloid-b protofibrils in biological samples, Journal of Neurochemistry, 2007, 103, 334-345
[6] Van Helmond et al, Higher Soluble Amyloid b Concentration in Frontal Cortex of Young Adults than in Normal Elderly or Alzheimer's Disease, Brain Pathology ISSN 1015-6305, doi:10.1111/j.1750-3639.2010.00374.x
[7] Selkoe et al., Isolation of Low-Molecular-Weight Proteins from Amyloid Plate Fibers in Alzheimer's Disease, Journal of Neurochemistry, (1986)
[8] Janssen et al., Signal loss due to oligomerization in ELISA analysis of amyloid-beta can be recovered by a novel sample pre-treatment method, MethodsX 2 (2015) 112-123
[9] Sun et al., Molecular Determinants and Genetic Modifiers of Aggregation and Toxicity for the ALS Disease Protein FUS/TLS, PLoS Biology, April 2011|Volume 9|Issue 4|e1000614
[10] Couthuis et al., Evaluating the role of the FUS/TLS-related gene EWSR1 in amyotrophic lateral sclerosis, Human Molecular Genetics, 2012, Vol. 21, No. 13 2899-2911
[11] Ryan et al., Ammonium hydroxide treatment of Aβ produces an aggregate free solution suitable for biophysical and cell culture characterization, (2013), PeerJ 1:e73; DOI 10.7717/peerj.73
[12] Dormann et al., Proteolytic processing of TAR DNA binding protein-43 by caspases produces C-terminal fragments with disease defining properties independent of progranulin, (2009), Journal of Neurochemistry, 110, 1082-1094
[13] Couthuis et al., A yeast functional screen predicts new candidate ALS disease genes, PNAS|Dec. 27, 2011|vol. 108|no. 52|20881-20890

1. An in vitro method for detecting in a sample a β-sheet aggregate form of a protein forming β-sheet aggregates (PAFβ), comprising the following steps:
a) In a first container:
a1) introducing a sample likely to contain a PAFβ,
a2) adjusting the pH to a pH ranging from 9.7 to 13.2 to disaggregate all or one portion of the PAFβ in order to obtain a β-sheet non-aggregate form of the protein forming β-sheet aggregates (PNAFβ),
a3) adjusting the pH to a pH ranging from 6 to 9,
b) Measuring the PNAFβ content in the first container with an appropriate immunological method;
c) In a second container:
c1) introducing the same sample as in step a1),
c2) adjusting the pH to a pH ranging from 6 to 9;
d) Measuring the PNAFβ content in the second container using the same method as in step b);
e) Comparing the contents measured in steps b) and d), a decrease in the content measured in step d) compared to the content measured in step b) indicating that the sample contains a PAFβ.

2. The method according to claim 1, wherein the protein forming β-sheet aggregates (PFβ) is selected from FUS (Fused in sarcoma), TAF15, EWSR1, DAZAP1, TIA-1, TTR (transthyretin), cystatin C, β2-microglobulin, beta amyloid peptide (such as β 1-40 amyloid peptide or β 1-42 amyloid peptide), TAU (Tubulin-Associated Unit), SOD1 (superoxide dismutase 1), α-synuclein, γ-synuclein, Huntingtin (HTT), prion and TDP-43 (TAR DNA-binding protein).

3. The method according to claim 1, wherein the PFβ is selected from beta amyloid 1-42, α-synuclein and TDP-43.

4. The method according to claim 1, wherein the sample comes from an individual having or being suspected of having a disease associated with PAFβ.

5. The method according to claim 1, wherein the sample comes from cells or tissue cultured in vitro.

6. The method according to claim 1, wherein the sample is selected from a blood sample, a plasma sample, a serum sample, a cerebrospinal fluid sample, a cell lysate, a cell homogenate, a tissue lysate or a tissue homogenate, such as a brain homogenate.

7. The method according to claim 1, wherein the sample is selected from a cell lysate, a cell homogenate, a tissue lysate, a tissue homogenate, a cell culture supernatant, a tissue culture supernatant, cellular sub-fractions or proteins (native or recombinant).

8. The method according to claim 1, wherein the pH in step a2) is adjusted with a base, such as NaOH.

9. The method according to claim 1, wherein the pH in step a3) is adjusted with an acid, such as HCl.

10. The method according to claim 1, wherein the pH is adjusted in step c2) with an acid/base mixture, such as a NaOH/HCl mixture.

11. The method according to claim 1, wherein the immunological method implemented in steps b) and d) uses:
a ligand capable of binding specifically to PNAFβ, said ligand being labeled with a tracer, or a pair of ligands capable of binding specifically to PNAFβ, at least one ligand of said pair of ligands being labeled with a tracer.

12. The method according to claim 11, wherein the immunological method implemented in steps b) and d) uses:
(i) a ligand capable of binding specifically to PNAFβ, said ligand being labeled with a tracer, wherein the ligand is selected from an antibody, an antibody fragment, a peptide or an aptamer; or
(ii) a pair of ligands capable of binding specifically to PNAFβ, at least one ligand of said pair of ligands being labeled with a tracer, wherein the pair of ligands is selected from a pair of antibodies, a pair of antibody fragments, a pair of peptides or a pair of aptamers, preferably a pair of antibodies.

13. The method according to claim 1, wherein the immunological method implemented in steps b) and d) is an ELISA method or a RET method.

14. The method according to claim 1, wherein steps b) and d) are carried out by a RET method and consist in:
(b1)/(d1) introducing into the container a first PNAFβ ligand labeled with a first member of a pair of RET partners and a second PNAFβ ligand labeled with a second member of the pair of RET partners, the pair of ligands being able to bind specifically to PNAFβ, and
(b2)/(d2) measuring the RET signal emitted in the container.

15. The method according to claim 1, wherein steps b) and d) are carried out by an ELISA method and consist in:
(b1)/(d1) introducing into the container, at the bottom of which a first PNAFβ ligand has previously been immobilized, a second PNAFβ ligand labeled with a tracer, the pair of ligands being capable of binding specifically to PNAFβ,
(b2)/(d2) measuring the ELISA signal emitted in the container.

16. An in vitro method for monitoring the therapeutic efficacy of a treatment for a disease associated with PAFβ in a patient, comprising the following steps:
A) Implementing the method according to claim 1 on a first sample of said patient, in which step e) consists in determining the ratio between the content measured in step b) and the content measured in step d) (“Ratio b)/d) of sample 1”);
B) Implementing the same method as in step A) on a second sample of said patient, to determine the “Ratio b)/d) of sample 2”;
C) Comparing the ratios determined in steps A) and B), in which therapeutic efficacy is observed when the ratio determined in step B) is lower than the ratio determined in step A).

17. An in vitro method for measuring the pharmacological efficacy of a drug molecule or a drug candidate on a disease associated with PAFβ in a test sample, comprising the following steps:
A) Implementing the method according to the invention on a first sample of said test sample, in which step e) consists in determining the ratio between the content measured in step b) and the content measured in step d) (“Ratio b)/d) of sample 1”);
B) Implementing the same method as in step A) on a second sample of said test sample, to determine the “Ratio b)/d) of sample 2”;
C) Comparing the ratios determined in steps A) and B), in which pharmacological efficacy is observed when the ratio determined in step B) is lower than the ratio determined in step A).
